SEQUENCE LISTING (75 sequences)

SEQ ID NO: 1 - protein, 5 aa, synthetic construct
  REGION 1..5: note = Synthetic
TYWIG

SEQ ID NO: 2 - protein, 16 aa, synthetic construct
  REGION 1..16: note = Synthetic
IIYPGDSDTRYSPPFQ

SEQ ID NO: 3 - protein, 8 aa, synthetic construct
  REGION 1..8: note = Synthetic
GNWNCFDY

SEQ ID NO: 4 - protein, 12 aa, synthetic construct
  REGION 1..12: note = Synthetic
RASQSVSSSYLA

SEQ ID NO: 5 - protein, 21 aa, synthetic construct
  REGION 1..21: note = Synthetic
GLYALASERARGARGALATHR

SEQ ID NO: 6 - protein, 24 aa, synthetic construct
  REGION 1..24: note = Synthetic
GLNARGTYRGLYSERSERHISTHR

SEQ ID NO: 7 - protein, 130 aa, synthetic construct
  REGION 1..130: note = Synthetic
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLA

SEQ ID NO: 8 - protein, 119 aa, synthetic construct
  REGION 1..119: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFP

SEQ ID NO: 9 - protein, 448 aa, synthetic construct
  REGION 1..448: note = Synthetic
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 214 aa, synthetic construct
  REGION 1..214: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEISRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 5 aa, synthetic construct
  REGION 1..5: note = Synthetic
SYDMS

SEQ ID NO: 12 - protein, 17 aa, synthetic construct
  REGION 1..17: note = Synthetic
LISGGGSQTYYAESVKG

SEQ ID NO: 13 - protein, 11 aa, synthetic construct
  REGION 1..11: note = Synthetic
PSGHYFYAMDV

SEQ ID NO: 14 - protein, 11 aa, synthetic construct
  REGION 1..11: note = Synthetic
RASQGISNWLA

SEQ ID NO: 15 - protein, 7 aa, synthetic construct
  REGION 1..7: note = Synthetic
AASSLQS

SEQ ID NO: 16 - protein, 9 aa, synthetic construct
  REGION 1..9: note = Synthetic
QQAESFPHT

SEQ ID NO: 17 - protein, 120 aa, synthetic construct
  REGION 1..120: note = Synthetic
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSS

SEQ ID NO: 18 - protein, 107 aa, synthetic construct
  REGION 1..107: note = Synthetic
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIK

SEQ ID NO: 19 - protein, 450 aa, synthetic construct
  REGION 1..450: note = Synthetic
EVQLLESGGGLVQPGGSLRLSCAASGFTFSSYDMSWVRQAPGKGLEWVSLISGGGSQTYYAESVKGRFTISRDNSKNTLYLQMNSLRAEDTAVYFCASPSGHYFYAMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPCEEQYGSTYRCVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 214 aa, synthetic construct
  REGION 1..214: note = Synthetic
DIQMTQSPSSVSASVGDRVTITCRASQGISNWLAWYQQKPGKAPKLLIFAASSLQSGVPSRFSGSGSGTDFTLTISSLQPEDFATYYCQQAESFPHTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, synthetic construct
  REGION 1..5: note = Synthetic
SYAMS

SEQ ID NO: 22 - protein, 17 aa, synthetic construct
  REGION 1..17: note = Synthetic
GITGSGGSTYYADSVKG

SEQ ID NO: 23 - protein, 13 aa, synthetic construct
  REGION 1..13: note = Synthetic
DPGTTVIMSWFDP

SEQ ID NO: 24 - protein, 12 aa, synthetic construct
  REGION 1..12: note = Synthetic
RASQSVRGRYLA

SEQ ID NO: 25 - protein, 7 aa, synthetic construct
  REGION 1..7: note = Synthetic
GASSRAT

SEQ ID NO: 26 - protein, 9 aa, synthetic construct
  REGION 1..9: note = Synthetic
QQYGSSPRT

SEQ ID NO: 27 - protein, 122 aa, synthetic construct
  REGION 1..122: note = Synthetic
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 28 - protein, 108 aa, synthetic construct
  REGION 1..108: note = Synthetic
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 29 - protein, 448 aa, synthetic construct
  REGION 1..448: note = Synthetic
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 30 - protein, 215 aa, synthetic construct
  REGION 1..215: note = Synthetic
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 453 aa, synthetic construct
  REGION 1..453: note = Synthetic
QVQLVESGGGVVQPGRSLRLSCVASGFTFSSYGMHWIRQAPGKGLEWVAVIWYEGSNKYYAESVKGRFTISRDNSKNTLYLQMNSLRAEDTAVYYCARGGRLGKDYYSGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, synthetic construct
  REGION 1..214: note = Synthetic
DIQMTQSPSSLSASVGDRVTITCRASQGIRNDLGWYQQKPGKAPKRLIYATSSLQSGVPSRFSGSGSGTEFTLTISSLQPEDFATYYCLQHNTYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 114 aa, synthetic construct
  REGION 1..114: note = Synthetic
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 34 - protein, 114 aa, synthetic construct
  REGION 1..114: note = Synthetic
NWIDVRYDLEKIESLIQSIHIDTTLYTDSDFHPSCKVTAMNCFLLELQVILHEYSNMTLNETVRNVLYLANSTLSSNKNVAESGCKECEELEEKTFTEFLQSFIRIVQMFINTS

SEQ ID NO: 35 - protein, 114 aa, synthetic construct
  REGION 1..114: note = Synthetic
NWIDVRYDLEKIESLIQFIHIDTTLYTDSDFHPSCKVTAMNCFLLELQVILHEYSNMTLNETVRNVLYLANSTLSSNKNVIESGCKECEELEERNFTEFLQSFIHIVQMFINTS

SEQ ID NO: 36 - protein, 114 aa, synthetic construct
  REGION 1..114: note = Synthetic
NWVNVISDLKKIEDLIQSMHIDATLYTESDVHPSCKVTAMKCFLLELQVISHESGDTDIHDTVENLIILANNILSSNGNITESGCKECEELEEKNIKEFLQSFVHIVQMFINTS

SEQ ID NO: 37 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWAMDYWGQGTLVTVSS

SEQ ID NO: 38 - protein, 122 aa, synthetic construct; identical to SEQ ID NO: 37
  REGION 1..122: note = Synthetic

SEQ ID NO: 39 - protein, 121 aa, synthetic construct
  REGION 1..121: note = Synthetic
VRLMASGGGLVQPGGSLRLSCAASEFTFSNYAMSWVRQAPGKGLEWVSTISRGGDYTYYPDSVKGRFTISRDNSKNTLYLQMNSLRAEDTAVYYCARRVSMITGGWAMDYWGQGTLVTVSS

SEQ ID NO: 40 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSLITGGWAMDYWGQGTLVTVSS

SEQ ID NO: 41 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPESVKGRFTISRDNSKNTLYLQMNSLRAEDTAVYYCARRVSFITGGWAMDYWGQGTLVTVSS

SEQ ID NO: 42 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSIITGGWAMDYWGQGTLVTVSS

SEQ ID NO: 43 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSAITGGWAMDYWGQGTLVTVSS

SEQ ID NO: 44 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSMITGGYAMDYWGQGTLVTVSS

SEQ ID NO: 45 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSMITGGFAMDYWGQGTLVTVSS

SEQ ID NO: 46 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSMITGGAAMDYWGQGTLVTVSS

SEQ ID NO: 47 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWALDYWGQGTLVTVSS

SEQ ID NO: 48 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWAFDYWGQGTLVTVSS

SEQ ID NO: 49 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGWAIDYWGQGTLVTVSS

SEQ ID NO: 50 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPESVKGRFTISRDNSKNTLYLQMNSLRAEDTAVYYCARRVSMITGGWAMDYWGQGTLVTVSS

SEQ ID NO: 51 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDTVKGRFTISRDNSKNTLYLQMNSLRAEDTAVYYCARRVSMITGGWAMDYWGQGTLVTVSS

SEQ ID NO: 52 - protein, 122 aa, synthetic construct
  REGION 1..122: note = Synthetic
EVQLVESGGGLVQPGGSLRLSCAASEFTFSNYAMSWVRQAPGKGLEWVATISRGGDYTYYPDSVKGRFTISRDNSKNTLYLQMNSLRAEDTAVYYCARRVSFITGGYAFDYWGQGTLVTVSS

SEQ ID NO: 53 - protein, 122 aa, synthetic construct
  REGION 1..122: note = Synthetic
EVQLVESGGGLVQPGGSLRLSCAASEFTFSNYAMSWVRQAPGKGLEWVATISRGGDYTYYPDSVKGRFTISRDNSKNTLYLQMNSLRAEDTAVYYCARRVSMITGGYAMDYWGQGTLVTVSS

SEQ ID NO: 54 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPDSVKGRFTISRDNSKNTLYLQMNSLRAEDTAVYYCARRVSFITGGYAMDYWGQGTLVTVSS

SEQ ID NO: 55 - protein, 122 aa, synthetic construct
  REGION 1..122: note = Synthetic
EVRLMASGGGLVQPGGSLRLSCAASEFTFSNYAMSWVRQAPGKGLEWVATISRGGDYTYYPESVKGRFTISRDNSKNTLYLQMNSLRAEDTAVYYCARRVSFITGGYAMDYWGQGTLVTVSS

SEQ ID NO: 56 - protein, 111 aa, synthetic construct
  REGION 1..111: note = Synthetic
VVMTQSPLSLPVTLGQPASISCRSSQSIVDITGNTYLEWYQQRPGQSPRLLIYKVFNRFSGVPDRFSGSGSGTDFTLKISRVEAEDVGVYYCFQDSFVPYTFGQGTKLEIK

SEQ ID NO: 57 - protein, 112 aa, synthetic construct
  REGION 1..112: note = Synthetic
DVVMTQSPLSLPVTLGQPASISCRSSQSIVDITGNTYLEWFQQRPGQSPRLLIYKVFNRFSGVPDRFSGSGSGTDFTLKISRVEAEDVGVYYCFQDSFVPYTFGQGTKLEIK

SEQ ID NO: 58 - protein, 112 aa, synthetic construct
  REGION 1..112: note = Synthetic
DVVMTQSPLSLPVTLGQPASISCRSSQSIVDITGNTYLEWYQQRPGQSPRRLIYKVFNRFSGVPDRFSGSGSGTDFTLKISRVEAEDVGVYYCFQDSFVPYTFGQGTKLEIK

SEQ ID NO: 59 - protein, 112 aa, synthetic construct
  REGION 1..112: note = Synthetic
DVVMTQSPLSLPVTLGQPASISCRSSQSIVDITGNTYLEWYQQRPGQSPRLLIYKVFNRFSGVPDRFSGSGSGTDFTLKISRVEAEDVGVYYCFQESFVPYTFGQGTKLEIK

SEQ ID NO: 60 - protein, 112 aa, synthetic construct
  REGION 1..112: note = Synthetic
DVVMTQSPLSLPVTLGQPASISCRSSQSIVDITGNTYLEWYQQRPGQSPRLLIYKVFNRFSGVPDRFSGSGSGTDFTLKISRVEAEDVGVYYCFQDTFVPYTFGQGTKLEIK

SEQ ID NO: 61 - protein, 112 aa, synthetic construct
  REGION 1..112: note = Synthetic
DVVMTQSPLSLPVTLGQPASISCRSSQSIVDITGNTYLEWFQQRPGQSPRRLIYKVFNRFSGVPDRFSGSGSGTDFTLKISRVEAEDVGVYYCFQESFVPYTFGQGTKLEIK

SEQ ID NO: 62 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 107 aa, synthetic construct [sequence omitted]

SEQ ID NO: 64 - protein, 122 aa, synthetic construct
  REGION 1..122: note = Synthetic
EVRLMASGGGLVKPGGSLKLSCAASEFTFSNYAMSWVRQTPEKRLEWVATISRGGDYTYYPDSVKGRFTISRDNAKNTLYLQMSSLRSEDTALYYCARRVSMITGGWAMDYWGQGTSVTVSS

SEQ ID NO: 65 - protein, 122 aa, synthetic construct
  REGION 1..122: note = Synthetic
EVRLLASGGGLVKPGGSLKLSCAASEFTFSNYAMSWVRQTPEKRLEWVATISRGGDYTYYPDSVKGRFTISRDNAKNTLYLQMSSLRSEDTALYYCARRVSMITGGWAMDYWGQGTSVTVSS

SEQ ID NO: 66 - protein, 122 aa, synthetic construct
  REGION 1..122: note = Synthetic
EVQLLASGGGLVKPGGSLKLSCAASEFTFSNYAMSWVRQTPEKRLEWVATISRGGDYTYYPDSVKGRFTISRDNAKNTLYLQMSSLRSEDTALYYCARRVSMITGGWAMDYWGQGTSVTVSS

SEQ ID NO: 67 - protein, 122 aa, synthetic construct
  REGION 1..122: note = Synthetic
EVRLMESGGGLVKPGGSLKLSCAASEFTFSNYAMSWVRQTPEKRLEWVATISRGGDYTYYPDSVKGRFTISRDNAKNTLYLQMSSLRSEDTALYYCARRVSMITGGWAMDYWGQGTSVTVSS

SEQ ID NO: 68 - protein, 112 aa, synthetic construct
  REGION 1..112: note = Synthetic
DVLMTQTPLSLPVSLGDQASISCRSSQSIVDITGNTYLEWYLQKPGQSPKLLIYKVFNRFSGVPDRFSGSGSGTDFTLKISRVEAEDLGLYYCFQDSFVPYTFGGGTKLEIK

SEQ ID NO: 69 - protein, 112 aa, synthetic construct
  REGION 1..112: note = Synthetic
EVVMTQSPATLSLSPGERATLSCRSSQSIVDITGNTYLEWYQQKPGQAPRLLIYKVFNRFSGIPARFSGSGSGTDFTLTISSLEPEDFAVYYCFQDSFVPYTFGQGTKLEIK

SEQ ID NO: 70 - protein, 153 aa, synthetic construct
  REGION 1..153: note = Synthetic
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 71 - protein, 107 aa, synthetic construct
  REGION 1..107: note = Synthetic
EIVLTQSPGTLSLSPGERATLSCRASQSVSSSYLAWYQQKPGQAPRLLIYGASRRATGIPDRFSGSGSGTDFTLTISRLEPEDFAVYYCQRYGSSHTFGQGTKLEIS

SEQ ID NO: 72 - protein, 118 aa, synthetic construct
  REGION 1..118: note = Synthetic
EVQLVQSGAEVKKPGESLKISCKVSGYFFTTYWIGWVRQMPGKGLEYMGIIYPGDSDTRYSPSFQGQVTISADKSISTAYLQWSSLKASDTAMYYCARGGNWNCFDYWGQGTLVTVSS

SEQ ID NO: 73 - protein, 11 aa, synthetic construct
  REGION 1..11: note = Synthetic
AWYDRELSEWV

SEQ ID NO: 74 - protein, 16 aa, synthetic construct
  REGION 1..16: note = Synthetic
DPAAWPLQQSLAWFDP

SEQ ID NO: 75 - protein, 12 aa, synthetic construct
  REGION 1..12: note = Synthetic
DTVENLIILANN

(19) United States
(12) Patent Application Publication
(10) Pub. No.: US 2024/0343795 A1
(43) Pub. Date: Oct. 17, 2024

(54) ANTIBODY FORMULATIONS

(71) Applicant: AMGEN INC., Thousand Oaks, CA (US)

(72) Inventors: Nicole BALL, Thousand Oaks, CA (US); Christopher SLOEY, Newbury Park, CA (US); Alexis LUERAS, Camarillo, CA (US); Rulin QIAN, Newbury Park, CA (US)

(73) Assignee: AMGEN INC., Thousand Oaks, CA (US)

(21) Appl. No.: 18/682,531

(22) PCT Filed: Aug. 11, 2022
(86) PCT No.: PCT/US2022/040056

§ 371 Date: Feb. 9, 2024

Related U.S. Application Data
(60) Provisional application No. 63/316,604, filed on Mar. 4, 2022.
(60) Provisional application No. 63/232,299, filed on Aug. 12, 2021.

(51) Int. Cl. C07K 16/24; A61K 39/00; A61K 47/12; A61K 47/18; A61K 47/26

(52) U.S. Cl. CPC C07K 16/244; A61K 47/12; A61K 47/183; A61K 47/26; A61K 2039/505; A61K 2039/545; C07K 2317/565

(57) ABSTRACT

Provided herein are liquid compositions comprising a high concentration of a monoclonal antibody, e.g., greater than about 100 mg/mL, which demonstrate storage-stability and reduced viscosity. In exemplary embodiments, the liquid composition comprises about less than about 400 mM arginine glutamate, and, in alternative exemplary embodiments, the liquid composition comprises proline and a buffer.

### CROSS REFERENCE TO RELATED APPLICATIONS

The benefit under 35 U.S.C. § 119(e) of U.S. Provisional Patent Application No. 63/232,299, filed on Aug. 12, 2021, and U.S. Provisional Patent Application No. 63/316,604, filed on Mar. 4, 2022, is hereby claimed, and the disclosure thereof is hereby incorporated by reference herein

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 99.1 KB file named “A-2756-WO01-SEC_SeqListing.xml”; created on Jul. 19, 2022.

### BACKGROUND

Increasing the concentration of therapeutic proteins in drug formulations can cause problems. For example, formulations comprising high concentrations of protein may lead to aggregation resulting in the formation of high molecular weight (HMW) species. HMW species, can be of concern in some protein formulations. Aggregation can also potentially affect the subcutaneous bioavailability and pharmacokinetics of a therapeutic protein and can cause a loss of bioactivity and increase in immunogenicity of the protein. High concentration protein formulations may result in elevated viscosity that can adversely impact drug product filling and administration.

Thus, there is a need in the art for high concentration formulations of antibodies with reduced viscosity, high stability and low levels of aggregation.

### SUMMARY

Presented herein are data demonstrating the initial stability, storage stability, and reduced viscosity of high concentration antibody liquid compositions comprising arginine glutamate or proline. Advantageously, the liquid compositions are isotonic and thus suitable for administration to subjects by injection or infusion. Surprisingly, the formulations presented herein comprise lower amounts of arginine glutamate or proline, compared to prior art formulations, and still retain the desired properties relating to initial aggregation, stability and viscosity. Accordingly, the present disclosure provides a liquid composition, e.g., an isotonic liquid composition, comprising a monoclonal antibody at a concentration greater than about 100 mg/mL, arginine glutamate, and a surfactant, wherein the pH of the liquid composition is about 4.5 to about 5.5. Also provided is a liquid composition, e.g., an isotonic liquid composition, comprising a monoclonal antibody at a concentration greater than about 100 mg/mL, proline, a buffer, and a surfactant, wherein the pH of the liquid composition is about 4.5 to about 5.5. In various aspects, the concentration of the monoclonal antibody is less than about 300 mg/mL or less than about 250 mg/mL. In exemplary embodiments, the presently disclosed liquid composition comprises about 110 mg/mL to about 250 mg/mL monoclonal antibody. In various aspects, the concentration of the monoclonal antibody is the concentration of the monoclonal antibody is about 120 mg/mL to about 180 mg/mL. Optionally, the liquid composition comprises about 120 mg/mL monoclonal antibody. In alternative aspects, the liquid composition comprises about 135 mg/mL to about 165 mg/mL monoclonal antibody, or about 140 mg/mL to about 160 mg/mL monoclonal antibody. In exemplary instances, the liquid composition comprises about 140 mg/mL or about 150 mg/mL monoclonal antibody. The monoclonal antibody of the presently disclosed liquid compositions is, in various aspects, an IgG₁ antibody. Alternatively, in various instances, the monoclonal antibody of the presently disclosed liquid compositions is an IgG₂ antibody. The monoclonal antibody, in exemplary aspects, is an anti-IL-15 antibody, an anti-PD-1 antibody, an anti-RANKL antibody, or an anti-GITR antibody. Exemplary antibodies are described herein. In various aspects, the liquid composition comprises about 200 mM to about 400 mM arginine glutamate. Optionally, the liquid composition comprises about 225 mM to about 350 mM arginine glutamate. In various instances, the liquid composition comprises about 250 mM to about 325 mM arginine glutamate, or about 2M00 mM to about 300 mM or about 200 mM arginine glutamate. In various aspects, the monoclonal antibody is formulated with about 50 mM to about 300 mM arginine base, optionally, about 85 mM to about 190 mM L-arginine. In various aspects, the monoclonal antibody is formulated with about 135 mM to about 165 mM arginine base. In various instances, the monoclonal antibody is formulated with about 155 mM to about 185 mM glutamic acid. In exemplary instances, the monoclonal antibody is formulated with about 150 mM arginine base and about 170 mM glutamic acid. In various instances, the monoclonal antibody is formulated with about 136 mM arginine base and about 159 mM glutamic acid. In various instances, the liquid composition comprises a molar ratio of arginine to glutamate of about 0.7:1.0 to about 1.1:1.0. For example, the molar ratio of arginine to glutamate is about 0.8:1.0 to about 1.1:1.0. In various aspects, arginine and glutamate are the only amino acids present in the liquid composition. In exemplary instances, the liquid composition comprises (a) about 150 mg to about 165 mg monoclonal antibody, (b) about 22 mg to about 26 mg L-arginine (e.g., about 24 mg to about 25 mg). (c) about 22 mg to about 26 mg glutamate (e.g., about 23 mg to about 24 mg), and (d) about 0.01 mg PS80, wherein the liquid composition has a pH of about 4.7 to about 5.3. In various instances, the liquid composition consists essentially of or consists of only (a) to (d). For example, the liquid composition does not comprise any additional buffer or any sugar. In alternative exemplary aspects, the liquid composition of the present disclosure comprises proline and a buffer, instead of arginine glutamate. In various aspects, the liquid composition comprises about 200 mM to about 300 mM proline, optionally, about 225 mM to about 275 mM proline or about 235 mM to about 265 mM. In various instances, the liquid composition comprises about 240 mM to about 260 mM proline. In various aspects, the proline is L-proline and/or proline is the only amino acid present in the liquid composition. In various instances, the buffer is selected from the group consisting of: succinate, glutamate, histidine, and acetate. Preferably, the buffer is acetate, e.g., acetate prepared with glacial acetic acid. In exemplary aspects, the buffer is prepared with about 1 mM to about 50 mM buffer, optionally, glacial acetic acid. In exemplary instances, the buffer is prepared with about 18 mM to about 22 mM buffer, optionally, 20 mM glacial acetic acid. Optionally, the liquid composition comprises about 30 mM to about 38 mM buffer, e.g., about 34 mM acetate. The pH of the buffer in various aspects is titrated with sodium hydroxide. In exemplary aspects, the surfactant is amphipathic and/or nonionic, optionally, a polysorbate. In exemplary instances, the surfactant is polysorbate 20 (PS20) or poly sorbate 80 (PS80) or a mixture thereof. The liquid composition in exemplary aspects comprises a surfactant at a concentration of about 0.001% (w/v) to about 0.050% (w/v). Optionally, the surfactant is present at a concentration of about 0.005% (w/v) to about 0.025% (w/v) or about 0.01% (w/v)±0.001% (w/v) surfactant. In preferred aspects, the liquid composition comprises about 0.01% (w/v) polysorbate 80 (PS80). The pH of the presently disclosed liquid composition is about 4.70 to about 5.30, optionally, about 5.0. In exemplary aspects, the liquid composition comprises, per mL liquid composition, (a) about 150 mg to about 165 mg monoclonal antibody, (b) about 22 mg to about 26 mg L-arginine, (c) about 22 mg to about 26 mg glutamate, and (d) about 0.01 mg PS80, wherein the liquid composition has a pH of about 4.7 to about 5.3. In various aspects, the liquid composition comprises, per mL liquid composition, about 150 mg to about 165 mg monoclonal antibody in about 180 mM to about 220 mM proline, about 30 mM to about 38 mM acetate, and about 0.01% (w/v) PS80, wherein the liquid composition has a pH of about 4.7 to about 5.3. In exemplary embodiments, the liquid composition is an isotonic liquid composition wherein less than about 5% of the antibody is degraded after storage at 2° C. to 8° C. for about 20 months to about 26 months, as determined by Size Exclusion Chromatography (SEC) and the liquid composition has a viscosity less than less than 30 cP at 25° C., 1000 s⁻¹.

Methods of preparing a liquid composition comprising a target concentration of a monoclonal antibody, wherein the target concentration is greater than about 100 mg/mL are further provided herein. In exemplary embodiments, the liquid composition is an isotonic liquid composition wherein less than about 5% of the antibody is degraded after storage at 2° C. to 8° C. for about 20 months to about 26 months, as determined by Size Exclusion Chromatography (SEC) and the liquid composition has a viscosity less than less than 30 cP at 25° C., 1000 s⁻¹. In exemplary embodiments, the method comprises (a) combining the monoclonal antibody with a diafiltration (DF) buffer comprising (i) about 50 mM to about 300 mM arginine base and (ii) an amount of glutamate to achieve a molar ratio of arginine to glutamate of about 0.7:1.0 to about 1.1:1.0 (e.g., about 0.8:1.0 to about 1.1:1.0), and (b) adding a surfactant. Optionally, the DF buffer comprises about 85 mM to about 190 mM arginine. Optionally, the DF buffer comprises about 135 mM to about 165 mM arginine base. In preferred aspects, the DF buffer comprises about 155 mM to about 185 mM glutamate, e.g., about 150 mM arginine base and about 170 mM glutamate. In various instances, the pH of the DF buffer is about the same as the final pH of the prepared liquid composition. Optionally, the final pH of the prepared liquid composition is about 4.5 to about 5.5, optionally, about 4.7 to about 5.3. In various instances, the surfactant is PS80 and/or is present at a final concentration of about 0.01% (w/v). A liquid composition prepared by the presently disclosed method is provided herein.

The present disclosure additionally provides an article of manufacture comprising any one of the presently disclosed isotonic liquid compositions. Optionally, the article comprises about 1 mL to about 5 mL of the isotonic liquid composition.

Also provided is a pre-filled syringe comprising any one of the presently disclosed isotonic liquid compositions, optionally, comprising about 1 mL to about 5 mL of the isotonic liquid composition.

A vial comprising any one of the presently disclosed isotonic liquid compositions is further provided by the present disclosure. Optionally, the vial comprises about 1 mL to about 5 mL of the isotonic liquid composition.

The present disclosure also provides an autoinjector comprising any one of the presently disclosed isotonic liquid compositions.

Methods of treating a disease in a subject are provided by the present disclosure. In exemplary embodiments, the method comprises administering to the subject an isotonic liquid composition of any one of the preceding claims in an amount effective to treat the disease. Additionally, use of the isotonic liquid composition of the present disclosure for treating a disease is provided.

### BRIEF DESCRIPTION OF THE DRAWINGS

FIG. 1 is a graph of the % HMW species formed in the indicated antibody formulations having the indicated target antibody concentrations.

### DETAILED DESCRIPTION

The present disclosure provides liquid compositions comprising high concentrations of an antibody, e.g., a monoclonal antibody. Collectively, antibodies form a family of plasma proteins known as immunoglobulins and comprise of immunoglobulin domains. (Janeway et al., Immunobiology: The Immune System in Health and Disease, 4^{th} ed., Elsevier Science Ltd/Garland Publishing, 1999. As used herein, the term “antibody” refers to a protein having a conventional immunoglobulin format, comprising heavy and light chains, and comprising variable and constant regions. For example, an antibody may be an IgG which is a “Y-shaped” structure of two identical pairs of polypeptide chains, each pair having one “light” (typically having a molecular weight of about 25 kDa) and one “heavy” chain (typically having a molecular weight of about 50-70 kDa). An antibody has a variable region and a constant region. In IgG formats, the variable region is generally about 100-110 or more amino acids, comprises three complementarity determining regions (CDRs), is primarily responsible for antigen recognition, and substantially varies among other antibodies that bind to different antigens. The constant region allows the antibody to recruit cells and molecules of the immune system. The variable region is made of the N-terminal regions of each light chain and heavy chain, while the constant region is made of the C-terminal portions of each of the heavy and light chains. (Janeway et al., “Structure of the Antibody Molecule and the Immunoglobulin Genes”, Immunobiology: The Immune System in Health and Disease, 4^{th} ed. Elsevier Science Ltd./Garland Publishing, (1999)).

The general structure and properties of CDRs of antibodies have been described in the art. Briefly, in an antibody scaffold, the CDRs are embedded within a framework in the heavy and light chain variable region where they constitute the regions largely responsible for antigen binding and recognition. A variable region typically comprises at least three heavy or light chain CDRs (Kabat et al., 1991, Sequences of Proteins of Immunological Interest, Public Health Service N.I.H., Bethesda, Md.; see also Chothia and Lesk, 1987, J. Mol. Biol. 196:901-917; Chothia et al., 1989, Nature 342: 877-883), within a framework region (designated framework regions 1-4, FR1, FR2, FR3, and FR4, by Kabat et al., 1991; see also Chothia and Lesk, 1987, supra).

Antibodies can comprise any constant region known in the art. Human light chains are classified as kappa and lambda light chains. Heavy chains are classified as mu, delta, gamma, alpha, or epsilon, and define the antibody's isotype as IgM, IgD, IgG, IgA, and IgE, respectively. IgG has several subclasses, including, but not limited to IgG1, IgG2, IgG3, and IgG4. IgM has subclasses, including, but not limited to, IgM1 and IgM2. Embodiments of the present disclosure include all such classes or isotypes of antibodies. The light chain constant region can be, for example, a kappa- or lambda-type light chain constant region, e.g., a human kappa- or lambda-type light chain constant region. The heavy chain constant region can be, for example, an alpha-, delta-, epsilon-, gamma-, or mu-type heavy chain constant regions, e.g., a human alpha-, delta-, epsilon-, gamma-, or mu-type heavy chain constant region. Accordingly, in exemplary embodiments, the antibody is an antibody of isotype IgA, IgD, IgE, IgG, or IgM, including any one of IgG1, IgG2, IgG3 or IgG4.

The antibody can be a monoclonal antibody, in exemplary instances. Accordingly, the present disclosure provides a liquid composition comprising a monoclonal antibody. Alternatively, the liquid composition may be a polyclonal antibody composition. In some embodiments, the antibody comprises a sequence that is substantially similar to a naturally-occurring antibody produced by a mammal, e.g., mouse, rabbit, goat, horse, chicken, hamster, human, and the like. In this regard, the antibody can be considered as a mammalian antibody, e.g., a mouse antibody, rabbit antibody, goat antibody, horse antibody, chicken antibody, hamster antibody, human antibody, and the like. In certain aspects, the antibody is a human antibody. In certain aspects, the antibody is a chimeric antibody or a humanized antibody. The term “chimeric antibody” refers to an antibody containing domains from two or more different antibodies. A chimeric antibody can, for example, contain the constant domains from one species and the variable domains from a second, or more generally, can contain stretches of amino acid sequence from at least two species. A chimeric antibody also can contain domains of two or more different antibodies within the same species. The term “humanized” when used in relation to antibodies refers to antibodies having at least CDR regions from a non-human source which are engineered to have a structure and immunological function more similar to true human antibodies than the original source antibodies. For example, humanizing can involve grafting a CDR from a non-human antibody, such as a mouse antibody, into a human antibody. Humanizing also can involve select amino acid substitutions to make a non-human sequence more similar to a human sequence.

In exemplary aspects, the antibody comprises (a) a heavy chain (HC) complementarity-determining region (CDR) 1 amino acid sequence set forth in Table A or a variant sequence thereof which differs by only 14 amino acids (e.g., 1, 2, 3, 4 amino acids) or which has at least or about 90% sequence identity; (b) an HC CDR2 amino acid sequence set forth in Table A or a variant sequence thereof which differs by only 14 amino acids or which has at least or about 90% sequence identity; (c) an HC CDR3 amino acid sequence set forth in Table A or a variant sequence thereof which differs by only 14 amino acids or which has at least or about 90% sequence identity; (d) a light chain (LC) CDR1 amino acid sequence set forth in Table A or a variant sequence thereof which differs by only 1-4 amino acids or which has at least or about 90% sequence identity; (e) an LC CDR2 amino acid sequence set forth in Table A or a variant sequence thereof which differs by only 14 amino acids or which has at least or about 90% sequence identity; (f) an LC CDR3 amino acid sequence set forth in Table A or a variant sequence thereof which differs by only 14 amino acids or which has at least or about 90% sequence identity; or (g) a combination of any two, three, four, five, or six of (a)-(f). In exemplary aspects, the antibody comprises a LC CDR1 amino acid sequence, a LC CDR2 amino acid sequence, and a LC CDR3 amino acid sequence set forth in Table A and at least 1 or 2 of the HC CDR amino acid sequences set forth in Table A. In exemplary aspects, the antibody comprises a HC CDR1 amino acid sequence, a HC CDR2 amino acid sequence, and a HC CDR3 amino acid sequence set forth in Table A and at least 1 or 2 of the LC CDR amino acid sequences set forth in Table A. In some embodiments, the antibody comprises all three such CDRs. In exemplary embodiments, the antibody comprises 3, 4, 5, or all 6 of the CDR amino acid sequences designated by the SEQ ID NOs: in a single column of Table A. In exemplary instances, the antibody comprises the HC CDR1, HC CDR2, HC CDR3, LC CDR1, LC CDR2, LC CDR3 of SEQ ID NOs: 1-6, respectively. In various instances, the antibody comprises the HC CDR1, HC CDR2, HC CDR3, LC CDR1, LC CDR2, LC CDR3 of SEQ ID NOs: 11-16, respectively. In various aspects, the antibody comprises the HC CDR1, HC CDR2, HC CDR3, LC CDR1, LC CDR2, LC CDR3 of SEQ ID NOs: 21-26, respectively. In exemplary instances, the antibody comprises the HC CDR1, HC CDR2, HC CDR3, LC CDR1, LC CDR2, LC CDR3 of SEQ ID NOs: 31-36, respectively. In exemplary aspects, the antibody comprises the HC variable (var) region sequence and/or the LC var region sequence as shown in Table A. In exemplary aspects, the antibody comprises the HC var region sequence of SEQ ID NO: 7 and/or the LC var region sequence of SEQ ID NO: 8, the antibody comprises the HC var region sequence of SEQ ID NO: 17 and/or the LC var region sequence of SEQ ID NO: 18, the antibody comprises the HC var region sequence of SEQ ID NO: 27 and/or the LC var region sequence of SEQ ID NO: 28. In exemplary instances, the antibody comprises the full-length (FL) HC sequence and/or the FL LC sequence shown in Table A. In exemplary aspects, the antibody comprises the FL HC of SEQ ID NO: 9 and/or the FL LC of SEQ ID NO: 10, the FL HC of SEQ ID NO: 19 and/or the FL LC of SEQ ID NO: 20, the FL HC of SEQ ID NO: 29 and/or the FL LC of SEQ ID NO: 30, or the FL HC of SEQ ID NO: 31 and/or the FL LC of SEQ ID NO: 32.

TABLE A

| | Antibody 1 | Antibody 2 | Antibody 3 | Antibody 4 |
|---|---|---|---|---|
| HC CDR1 | 1 | 11 | 21 | |
| HC CDR2 | 2 | 12 | 22 | |
| HC CDR3 | 3 | 13 | 23 | |
| LC CDR1 | 4 | 14 | 24 | |
| LC CDR2 | 5 | 15 | 25 | |
| LC CDR3 | 6 | 16 | 26 | |
| HC var | 7 | 17 | 27 | |
| LC var | 8 | 18 | 28 | |
| FL HC | 9 | 19 | 29 | 31 |
| FL LC | 10 | 20 | 30 | 32 |

In exemplary aspects, the antibody is an anti-IL-15 described in the art. For example, the antibody is, in various aspects, an anti-IL-15 antibody described in U.S. Pat. No. 10,301,384, which is incorporated herein by reference. In various aspects, the antibody comprises (i) a heavy chain variable region selected from: SEQ ID NO: 37, SEQ ID NO: 38, SEQ ID NO: 44, SEQ ID NO: 53, SEQ ID NO: 39, SEQ ID NO: 40, SEQ ID NO: 41, SEQ ID NO: 42, SEQ ID NO: 43. SEQ ID NO: 45, SEQ ID NO: 46, SEQ ID NO: 47, SEQ ID NO: 48, SEQ ID NO: 49, SEQ ID NO: 50, SEQ ID NO: 51. SEQ ID NO: 52, SEQ ID NO: 54 and SEQ ID NO: 55, and (ii) a light chain variable region selected from: SEQ ID NO: 56, SEQ ID NO: 57, SEQ ID NO: 58, SEQ ID NO: 59, SEQ ID NO: 60, and SEQ ID NO: 61. Optionally, the antibody comprises the amino acid sequence of any one of SEQ ID NOs: 62-64, 68, 71, and 72.

### Antibody Concentration

In exemplary aspects, the presently disclosed composition comprises the antibody at a concentration greater than about 100 mg/mL and, optionally, less than about 450 mg/mL, less than about 400 mg/mL, less than about 350 mg/mL, less than about 300 mg/mL or less than about 250 mg/mL. In various instances, the antibody is present in the composition at a concentration of about 110 mg/mL to about 300 mg/mL, e.g., about 110 mg/mL to about 290 mg/mL, about 110 mg/mL to about 280 mg/mL, about 110 mg/mL to about 270 mg/mL, about 110 mg/mL to about 260 mg/mL, about 110 mg/mL to about 250 mg/mL, about 110 mg/mL to about 240 mg/mL, about 110 mg/mL to about 230 mg/mL, about 110 mg/mL to about 220 mg/mL, about 110 mg/mL to about 210 mg/mL, about 110 mg/mL to about 200 mg/mL, about 110 mg/mL to about 190 mg/mL, about 110 mg/mL to about 180 mg/mL, about 110 mg/mL to about 170 mg/mL, about 110 mg/mL to about 160 mg/mL, about 110 mg/mL to about 150 mg/mL, about 110 mg/mL to about 140 mg/mL, about 110 mg/mL to about 130 mg/mL, about 110 mg/mL to about 120 mg/mL, about 120 mg/mL to about 300 mg/mL, about 130 mg/mL to about 300 mg/mL, about 140 mg/mL to about 300 mg/mL, about 150 mg/mL to about 300 mg/mL, about 160 mg/mL to about 300 mg/mL, about 170 mg/mL to about 300 mg/mL, about 180 mg/mL to about 300 mg/mL, about 190 mg/mL to about 300 mg/mL, about 200 mg/mL to about 300 mg/mL, about 210 mg/mL to about 300 mg/mL, about 220 mg/mL to about 300 mg/mL, about 230 mg/mL to about 300 mg/mL, about 240 mg/mL to about 3M00 mg/mL, about 250 mg/mL to about 300 mg/mL, about 260 mg/mL to about 300 mg/mL, about 270 mg/mL to about 3M00 mg/mL, about 280 mg/mL to about 300 mg/mL, or about 290 mg/mL to about 300 mg/mL. In various instances, the concentration of the antibody is about 120 mg/mL to about 250 mg/mL, optionally, about 120 mg/mL to about 240 mg/mL, about 120 mg/mL to about 230 mg/mL, about 120 mg/mL to about 220 mg/mL, about 120 mg/mL to about 210 mg/mL, about 120 mg/mL to about 200 mg/mL, about 120 mg/mL to about 190 mg/mL, about 120 mg/mL to about 180 mg/mL, about 120 mg/mL to about 170 mg/mL, about 120 mg/mL to about 160 mg/mL, about 120 mg/mL to about 150 mg/mL, about 120 mg/mL to about 140 mg/mL, about 120 mg/mL to about 130 mg/mL, about 130 mg/mL to about 250 mg/mL, about 140 mg/mL to about 250 mg/mL, about 150 mg/mL to about 250 mg/mL, about 160 mg/mL to about 250 mg/mL, about 170 mg/mL to about 250 mg/mL, about 180 mg/mL to about 250 mg/mL, about 190 mg/mL to about 250 mg/mL, about 200 mg/mL to about 250 mg/mL, about 210 mg/mL to about 250 mg/mL, about 220 mg/mL to about 250 mg/mL, about 230 mg/mL to about 250 mg/mL, or about 240 mg/mL to about 250 mg/mL. In various instances, the concentration of the antibody in the liquid composition is about 160 mg/mL to about 250 mg/mL, e.g., about 180 mg/mL to about 225 mg/mL, or about 180 mg/mL to about 200 mg/mL. In some aspects, the antibody is present in the composition at a concentration of about 130 mg/mL to about 225 mg/mL, about 130 mg/mL to about 220 mg/mL or about 130 mg/mL to about 200 mg/mL. In various instances, the antibody is present in the composition at a concentration of about 135 mg/mL to about 165 mg/mL, optionally about 140 mg/mL to about 160 mg/mL. In exemplary aspects, the liquid composition comprises about 120 mg/mL antibody, about 140 mg/mL, about 150 mg/mL or about 165 mg/mL. In exemplary aspects, the liquid composition comprises about 150 mg/mL.

### Arginine Glutamate

In various instances, the liquid composition comprises greater than or about 10 mM to less than or about 450 mM arginine glutamate, e.g., less than about 440 mM, less than about 430 mM, less than 420 mM, less than 410 mM arginine glutamate. In various aspects, the liquid composition comprises about 10 mM to about 400 mM, about 25 mM to about 400 mM, about 50 mM to about 400 mM, about 75 mM to about 400 mM, about 100 mM to about 400 mM, about 125 mM to about 400 mM, about 150 mM to about 400 mM, about 200 mM to about 300 mM, about 200 mM to about 400 mM, about 225 mM to about 350 mM or about 400 mM, about 250 mM to about 325 mM or about 40 mM, about 275 mM to about 400 mM, about 300 mM to about 400 mM, about 325 mM to about 400 mM, about 350 mM to about 400 mM, about 375 mM to about 400 mM, about 10 mM to about 375 mM, about 10 mM to about 350 mM, about 10 mM to about 325 mM, about 10 mM to about 300 mM, about 10 mM to about 275 mM, about 10 mM to about 250 mM, about 10 mM to about 225 mM, about 10 mM to about 200 mM, about 10 mM to about 175 mM, about 10 mM to about 150 mM, about 10 mM to about 125 mM, about 10 mM to about 100 mM, about 10 mM to about 75 mM, about 10 mM to about 50 mM, or about 10 mM to about 25 mM arginine glutamate. In exemplary aspects, the liquid composition comprises at least or about 50 mM, at least or about 75 mM, at least or about 100 mM arginine glutamate and/or less than about 450 mM, less than about 400 mM, less than about 350 mM, less than about 300 mM, less than about 250 mM or less than about 200 mM arginine glutamate. In exemplary instances, the liquid composition comprises about 75 mM to about 250 mM, about 100 mM to about 200 mM, or about 125 mM to about 175 mM arginine glutamate. In various instances, the liquid composition comprises about 200 mM arginine glutamate. In exemplary aspects, the liquid composition comprises about 250 mM to about 350 mM, e.g., about 260 mM to about 350 mM, about 270 mM to about 350 mM, about 280 mM to about 350 mM, about 290 mM to about 350 mM, about 300 mM to about 350 mM, about 310 mM to about 350 mM, about 320 mM to about 350 mM, about 330 mM to about 350 mM, about 340 mM to about 350 mM, about 250 mM to about 340 mM, about 250 mM to about 330 mM, about 250 mM to about 320 mM, about 250 mM to about 310 mM, about 250 mM to about 300 mM, about 250 mM to about 290 mM, about 250 mM to about 280 mM, about 250 mM to about 270 mM, or about 250 mM to about 260 mM arginine glutamate. In exemplary aspects, the liquid composition comprising about 260 mM to about 340 mM or about 270 mM to about 330 mM arginine glutamate. In various aspects, arginine glutamate is a salt comprising arginine base and glutamic acid. In various aspects, arginine glutamate is a salt comprising L-arginine base and L-glutamic acid. In various instances, arginine glutamate is a salt of arginine comprising a glutamate counterion. For purposes herein, the term “glutamate” refers to glutamate, its conjugate acid (glutamic acid), or a combination thereof. In various aspects, the liquid composition comprises about 70 mM to about 210 mM arginine (also referred to herein as “arginine base”), and about 80 mM to about 240 mM glutamate. In various aspects, the composition comprises about 70 mM to about 210 mM arginine base and about 80 mM to about 240 mM glutamic acid. In various aspects, the liquid composition comprises about 100 mM to about 170 mM arginine base, optionally about 120 mM to about 150 mM arginine base. In various aspects, the arginine base is L-arginine base. In exemplary aspects, the liquid composition comprises about 136 mM arginine base. In exemplary aspects, the liquid composition comprises about 136 mM L-arginine base. In various aspects, the liquid composition comprises about 120 mM to about 200 mM glutamate, optionally about 140 mM to about 175 mM glutamate. In exemplary aspects, the liquid composition comprises about 159 mM glutamate. In various aspects, the presently disclosed composition comprises about 120 mM to about 200 mM glutamic acid, e.g., L-glutamic acid, optionally about 140 mM to about 175 mM glutamic acid, e.g., L-glutamic acid. In exemplary aspects, the composition comprises about 159 mM glutamic acid, e.g., L-glutamic acid. In various instances, the liquid composition comprises about 100 mM to about 200 mM arginine and about 100 mM to about 200 mM glutamate. Optionally, the liquid composition comprises about 125 mM to about 175 mM arginine or about 125 to about 150 mM arginine and about 125 mM to about 200 mM glutamate or about 140 mM to about 185 mM glutamate. In various instances, the liquid composition comprises about 136 mM arginine base and about 159 mM glutamate. In various instances, the composition comprises about 100 mM to about 200 mM arginine and about 100 mM to about 200 mM glutamic acid. Optionally, the composition comprises about 125 mM to about 175 mM arginine, e.g., L-arginine base, or about 125 to about 150 mM arginine, e.g., L-arginine base, and about 125 mM to about 200 mM glutamic acid, e.g., L-glutamic acid or about 140 mM to about 185 mM glutamic acid, e.g., L-glutamic acid. In various instances, the composition comprises about 136 mM L-arginine base and about 159 mM glutamic acid, e.g., L-glutamic acid. In various aspects, the composition comprises about 120 mM to about 200 mM glutamic acid, optionally about 140 mM to about 175 mM glutamic acid. In exemplary aspects, the composition comprises about 159 mM glutamic acid. In various instances, the composition comprises about 100 mM to about 200 mM L-arginine base and about 100 mM to about 200 mM L-glutamic acid. Optionally, the composition comprises about 125 mM to about 175 mM L-arginine base or about 125 to about 150 mM L-arginine base and about 125 mM to about 200 mM L-glutamic acid or about 140 mM to about 185 mM L-glutamic acid. In various instances, the composition comprises about 136 mM L-arginine base and about 159 mM L-glutamic acid.

In various aspects, the antibody is formulated with about 25 mM to about 190 mM arginine (e.g., L-arginine base) and about 25 mM to about 200 mM glutamic acid (e.g., L-glutamic acid). For purposes herein, the indicated amounts of excipients following “formulated with” refer to the amounts of the indicated excipients in a DF buffer used in preparing the liquid composition comprising the monoclonal antibody. Accordingly, an antibody formulated with about 25 mM to about 190 mM arginine and about 25 mM to about 200 mM glutamic acid, in various aspects, means that the DF buffer into which the antibody was exchanged or with which the antibody was combined comprised about 25 mM to about 190 mM arginine and about 25 mM to about 200 mM glutamic acid. In various instances, the antibody is formulated with about 50 mM to about 300 mM arginine or 85 mM to about 190 mM arginine. In various aspects, the antibody is formulated with 100 mM to about 180 mM arginine (e.g., about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 180 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 135 mM to about 155 mM or about 135 mM to about 165 mM) and about 110 mM to about 240 mM glutamic acid (e.g., about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 140 mM to about 160 mM, about 145 mM to about 155 mM glutamic acid or about 155 mM to about 185 mM glutamic acid). In various instances, the antibody is formulated with about 135 mM to about 165 mM arginine and about 145 mM to about 185 mM glutamic acid.

In various instances, the antibody is formulated in about 10 mM to about 125 mM arginine and about 25 mM to about 225 mM glutamate. For purposes herein, the indicated amounts of excipients following “formulated in” refer to the amounts of the indicated excipients in the liquid composition comprising the monoclonal antibody. Accordingly, an antibody formulated in about 10 mM to about 125 mM arginine and about 25 mM to about 225 mM glutamate, in various instances, means the liquid composition comprises the antibody and about 10 mM to about 125 mM arginine and about 25 mM to about 225 mM glutamate. In various aspects, the antibody is formulated in about 55 mM to about 135 mM arginine (e.g., about 55 mM to about 125 mM, about 55 mM to about 115 mM, about 55 mM to about 105 mM, about 55 mM to about 95 mM, about 55 mM to about 85 mM, about 55 mM to about 75 mM, about 55 mM to about 65 mM, about 65 mM to about 135 mM, about 75 mM to about 135 mM, about 85 mM to about 135 mM, about 95 mM to about 135 mM, about 105 mM to about 135 mM, about 115 mM to about 145 mM, about 125 mM to about 135 mM, about 75 mM to about 115 mM, about 85 mM to about 105 mM arginine) and about 130 mM to about 210 mM glutamate (e.g., about 130 mM to about 200 mM, about 130 mM to about 240 mM, about 130 mM to about 180 mM, about 130 mM to about 170 mM, about 130 mM to about 160 mM, about 130 mM to about 150 mM, about 130 mM to about 140 mM, about 140 mM to about 210 mM, about 150 mM to about 210 mM, about 160 mM to about 210 mM, about 170 mM to about 210 mM, about 180 mM to about 210 mM, about 190 mM to about 210 mM, about 200 mM to about 210 mM, about 150 mM to about 190 mM, about 160 mM to about 180 mM, about 165 mM to about 175 mM glutamate). In various instances, the composition comprises the antibody and about 10 mM to about 125 mM arginine base, e.g., L-arginine base and about 25 mM to about 225 mM glutamic acid, e.g., L-glutamic acid. In various aspects, the antibody is formulated in about 55 mM to about 135 mM L-arginine base (e.g., about 55 mM to about 125 mM, about 55 mM to about 115 mM, about 55 mM to about 105 mM, about 55 mM to about 95 mM, about 55 mM to about 85 mM, about 55 mM to about 75 mM, about 55 mM to about 65 mM, about 65 mM to about 135 mM, about 75 mM to about 135 mM, about 85 mM to about 135 mM, about 95 mM to about 135 mM, about 105 mM to about 135 mM, about 115 mM to about 145 mM, about 125 mM to about 135 mM, about 75 mM to about 115 mM, about 85 mM to about 105 mM L-arginine base) and about 130 mM to about 210 mM glutamic acid (e.g., about 130 mM to about 200 mM, about 130 mM to about 240 mM, about 130 mM to about 180 mM, about 130 mM to about 170 mM, about 130 mM to about 160 mM, about 130 mM to about 150 mM, about 130 mM to about 140 mM, about 140 mM to about 210 mM, about 150 mM to about 210 mM, about 160 mM to about 210 mM, about 170 mM to about 210 mM, about 180 mM to about 210 mM, about 190 mM to about 210 mM, about 200 mM to about 210 mM, about 150 mM to about 190 mM, about 160 mM to about 180 mM, about 165 mM to about 175 mM L-glutamic acid).

In various aspects, the antibody is formulated with about 70 mM to about 210 mM arginine and about 80 mM to about 240 mM glutamic acid. In various aspects, the antibody is formulated with about 100 mM to about 170 mM arginine base, optionally about 120 mM to about 150 mM arginine base. In exemplary aspects, the antibody is formulated with about 136 mM arginine base. In various aspects, the antibody is formulated with about 120 mM to about 200 mM glutamic acid, optionally about 140 mM to about 175 mM glutamic acid. In exemplary aspects, the antibody is formulated with about 159 mM glutamic acid. In various aspects, the glutamic acid is L-glutamic acid. In various instances, the antibody is formulated with about 100 mM to about 200 mM arginine and about 100 mM to about 200 mM glutamic acid. Optionally, the antibody is formulated with about 125 mM to about 175 mM arginine or about 125 to about 150 mM arginine and about 125 mM to about 200 mM glutamic acid or about 140 mM to about 185 mM glutamic acid. In various instances, the antibody is formulated with 150 mM arginine base and about 170 mM glutamic acid. In various aspects, the arginine glutamate present in the liquid composition is made with about 25 mM to about 190 mM arginine and about 25 mM to about 200 mM glutamic acid. In various aspects, the antibody is formulated with 100 mM to about 180 mM arginine (e.g., about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 180 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 16 mM, about 135 mM to about 155 mM) and about 110 mM to about 240 mM glutamic acid (e.g., about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 140 mM to about 160 mM, about 145 mM to about 155 mM glutamic acid). In various instances, the arginine glutamate present in the liquid composition is made with about 135 mM to about 145 mM arginine and about 145 mM to about 155 mM glutamic acid. In various aspects, the arginine glutamate present in the liquid composition is made with about 70 mM to about 210 mM arginine base and about 80 mM to about 240 mM glutamic acid. In various aspects, the arginine glutamate present in the liquid composition is made with about 100 mM to about 170 mM arginine base or about 120 mM to about 150 mM arginine base and about 120 mM to about 200 mM glutamic acid or about 140 mM to about 175 mM glutamic acid. In various aspects, the arginine glutamate present in the liquid composition is made with about 136 mM arginine base and about 159 mM glutamic acid.

In various instances, the antibody is formulated in about 10 mM to about 125 mM arginine and about 25 mM to about 225 mM glutamate. In various aspects, the liquid composition comprises about 55 mM to about 135 mM arginine (e.g., about 55 mM to about 125 mM, about 55 mM to about 115 mM, about 55 mM to about 105 mM, about 55 mM to about 95 mM, about 55 mM to about 85 mM, about 55 mM to about 75 mM, about 55 mM to about 65 mM, about 65 mM to about 135 mM, about 75 mM to about 135 mM, about 85 mM to about 135 mM, about 95 mM to about 135 mM, about 105 mM to about 135 mM, about 115 mM to about 145 mM, about 125 mM to about 135 mM, about 75 mM to about 115 mM, about 85 mM to about 105 mM arginine) and about 130 mM to about 210 mM glutamate (e.g., about 130 mM to about 200 mM, about 130 mM to about 240 mM, about 130 mM to about 180 mM, about 130 mM to about 170 mM, about 130 mM to about 160 mM, about 130 mM to about 150 mM, about 130 mM to about 140 mM, about 140 mM to about 210 mM, about 150 mM to about 210 mM, about 160 mM to about 210 mM, about 170 mM to about 210 mM, about 180 mM to about 210 mM, about 190 mM to about 210 mM, about 200 mM to about 210 mM, about 150 mM to about 190 mM, about 160 mM to about 180 mM, about 165 mM to about 175 mM glutamate). In various aspects, the antibody is formulated in about 70 mM to about 210 mM arginine base and about 80 mM to about 240 mM glutamate. In various aspects, the antibody is formulated in about 100 mM to about 170 mM arginine base or about 120 mM to about 150 mM arginine base and about 120 mM to about 200 mM glutamate or about 140 mM to about 175 mM glutamate. In various aspects, the antibody is formulated in about 136 mM arginine base and about 159 mM glutamate. In various instances, the antibody is formulated in about 10 mM to about 125 mM arginine and about 25 mM to about 225 mM glutamic acid. In various aspects, the liquid composition comprises about 55 mM to about 135 mM arginine (e.g., about 55 mM to about 125 mM, about 55 mM to about 115 mM, about 55 mM to about 105 mM, about 55 mM to about 95 mM, about 55 mM to about 85 mM, about 55 mM to about 75 mM, about 55 mM to about 65 mM, about 65 mM to about 135 mM, about 75 mM to about 135 mM, about 85 mM to about 135 mM, about 95 mM to about 135 mM, about 105 mM to about 135 mM, about 115 mM to about 145 mM, about 125 mM to about 135 mM, about 75 mM to about 115 mM, about 85 mM to about 105 mM L-arginine base) and about 130 mM to about 210 mM L-glutamic acid (e.g., about 130 mM to about 200 mM, about 130 mM to about 240 mM, about 130 mM to about 180 mM, about 130 mM to about 170 mM, about 130 mM to about 160 mM, about 130 mM to about 150 mM, about 130 mM to about 140 mM, about 140 mM to about 210 mM, about 150 mM to about 210 mM, about 160 mM to about 210 mM, about 170 mM to about 210 mM, about 180 mM to about 210 mM, about 190 mM to about 210 mM, about 200 mM to about 210 mM, about 150 mM to about 190 mM, about 160 mM to about 180 mM, about 165 mM to about 175 mM L-glutamic acid). In various aspects, the antibody is formulated in about 70 mM to about 210 mM arginine base and about 80 mM to about 240 mM glutamic acid. In various aspects, the antibody is formulated in about 100 mM to about 170 mM arginine base or about 120 mM to about 150 mM arginine base and about 120 mM to about 200 mM glutamic acid or about 140 mM to about 175 mM glutamic acid. In various aspects, the antibody is formulated in about 136 mM arginine base and about 159 mM glutamic acid.

In exemplary embodiments, the presently disclosed liquid composition comprises, per mL liquid composition, (a) about 150 mg to about 165 mg monoclonal antibody, (b) about 22 mg to about 26 mg L-arginine, (c) about 22 mg to about 26 mg glutamate, and (d) about 0.01 mg PS80, wherein the liquid composition has a pH of about 4.7 to about 5.3, and 0.01% (w/v) polysorbate 80. The arginine in various aspects is L-arginine. In exemplary aspects, the glutamate is L-glutamate. In exemplary embodiments, the presently disclosed composition comprises, per mL liquid composition, (a) about 150 mg to about 165 mg monoclonal antibody, (b) about 22 mg to about 26 mg L-arginine base, (c) about 22 mg to about 26 mg L-glutamic acid, and (d) about 0.01 mg PS80, wherein the liquid composition has a pH of about 4.7 to about 5.3, and 0.01% (w/v) polysorbate 80. Optionally, the pH is about 4.7 to about 5.3. In exemplary embodiments, the presently disclosed liquid composition comprises, per mL liquid composition, (i) about 135 mg to about 165 mg of an anti-IL-15 antibody comprising a Heavy Chain (HC) Complementarity-Determining Region (CDR) 1 of SEQ ID NO: 1; a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, (ii) about 21 mg to about 26 mg arginine base, (iii) about 21 mg to about 26 mg glutamate, and (iv) about 0.01 mg PS80, wherein the liquid composition has a pH of about 4.5 to about 5.5. Optionally, the pH is about 4.7 to about 5.3. The arginine in various aspects is L-arginine. In exemplary aspects, the glutamate is L-glutamate. In exemplary embodiments, the presently disclosed composition comprises (i) about 135 mg to about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, (ii) about 21 mg to about 26 mg L-arginine base, (iii) about 21 mg to about 26 mg L-glutamic acid, and (iv) about 0.01 mg PS80, wherein the liquid composition has a pH of about 4.5 to about 5.5. Optionally, the pH is about 4.7 to about 5.3. In exemplary embodiments, the presently disclosed liquid composition comprises, per mL liquid composition, (i) about 150 mg of ordesekimab (ii) about 23.6 mg arginine base, (iii) about 23.4 mg glutamate, and (iv) about 0.01% (w/v) polysorbate 80 (PS80), wherein the liquid composition has a pH of about 5.0. In exemplary embodiments, the presently disclosed composition comprises (i) about 150 mg of ordesekimab (ii) about 23.6 mg L-arginine base, (iii) about 23.4 mg L-glutamic acid, and (iv) about 0.01% (w/v) polysorbate 80 (PS80), wherein the liquid composition has a pH of about 5.0. Ordesekimab comprises HC CDR1-CDR3 of SEQ ID NOs: 1-3, respectively, and LC CDR1-CDR3 of SEQ ID NOs: 4-6, respectively. Ordesekimab comprises a heavy chain variable region of SEQ ID NO: 7 and a light chain variable region of SEQ ID NO: 8. Also, ordesekimab comprises a heavy chain of SEQ ID NO: 9 and a light chain of SEQ ID NO: 10. In exemplary embodiments, the presently disclosed liquid composition comprises, per mL liquid composition, about 150 mg or about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, formulated in a solution comprising about 21 mg to about 26 mg arginine base, about 21 mg to about 26 mg glutamate, and about 0.01 mg PS80, wherein the liquid composition has a pH of about 4.5 to about 5.5. In exemplary embodiments, the presently disclosed liquid composition comprises, per mL liquid composition, about 150 mg or about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, formulated with a solution comprising about 21 mg to about 26 mg arginine base, about 21 mg to about 26 mg glutamic acid, and about 0.01 mg PS80, wherein the liquid composition has a pH of about 4.5 to about 5.5.

### Proline

In exemplary embodiments, the liquid compositions of the present disclosure comprise proline, e.g., L-proline, D-proline. In exemplary aspects, the liquid compositions of the present disclosure comprise L-proline. In some aspects, proline is the only amino acid present in the composition. In various embodiments, the liquid composition comprises about 50 mM to about 400 mM proline or about 100 mM to about 350 mM proline. In exemplary aspects, the liquid compositions of the present disclosure comprise about 115 mM to about 345 mM proline. In exemplary instances, the liquid compositions of the present disclosure comprise about 170 mM to about 290 mM proline, optionally, about 200 mM to about 255 mM proline, e.g., about 230 mM proline. In exemplary aspects, the liquid compositions of the present disclosure comprise about 100 mM to about 300 mM L-proline. Optionally, the liquid composition comprises about 75 mM to about 400 mM, about 100 mM to about 400 mM, about 125 mM to about 400 mM, about 150 mM to about 400 mM, about 175 mM to about 400 mM, about 200 mM to about 400 mM, about 225 mM to about 400 mM, about 250 mM to about 400 mM, about 275 mM to about 400 mM, about 300 mM to about 400 mM, about 325 mM to about 400 mM, about 350 mM to about 400 mM, about 375 mM to about 400 mM, about 50 mM to about 375 mM, about 50 mM to about 350 mM, about 50 mM to about 325 mM, about 50 mM to about 300 mM, about 50 mM to about 275 mM, about 50 mM to about 250 mM, about 50 mM to about 225 mM, about 50 mM to about 200 mM, about 50 mM to about 175 mM, about 50 mM to about 150 mM, about 50 mM to about 125 mM, about 50 mM to about 100 mM, or about 50 mM to about 75 mM. In various instances, the concentration of proline in the liquid composition is about 200 mM to about 300 mM, about 225 mM to about 275 mM, about 235 mM to about 265 mM, or about 240 mM to about 260 mM. In various aspects, the liquid composition comprises at least about 50 mM or at least or about 100 mM and less than about 260 mM proline.

In exemplary embodiments, wherein the liquid composition comprises proline, the isotonic liquid composition also comprises a buffer. The buffer can be, for instance, an organic buffer. The buffer in some aspects, is centered at 25° C. around pH 4.0 to 6.0, or 4.5 to 5.5, or 4.2 or 5.7, for example. In various embodiments, the buffer can have a pKa within one pH unit of pH 5.0-5.2 at 25° C. One such buffer is acetic acid/acetate, having a pKa of about 4.75 at 25° C. Other alternative buffers contemplated include buffers based on ions including succinate (pKa of 4.21 at 25° C.), propionate (pKa of 4.87 at 25° C.), malate (pKa of 5.13 at 25° C.), pyridine (pKa of 5.23 at 25° C.) and piperazine (pKa of 5.33 at 25° C.). It is contemplated that the buffer can be provided as the sodium salt (or disodium salt, as appropriate), or in the alternative as a potassium, magnesium, or ammonium salt. In various aspects, the buffer is succinate, glutamate, histidine or acetate, or a combination thereof. Buffers based on acetate are particularly contemplated. In some aspects, the buffer is made with glacial acetic acid and optionally, the pH of the buffer is attained by adding sodium hydroxide until the target pH or final pH of the buffer is reached. In various aspects, the target pH or the final pH of the buffer is about 4.5 to about 5.5, optionally, about 4.7 to about 5.3, or about 5.0. In exemplary instances, the composition comprises about 1 mM to about 100 mM buffer. In exemplary instances, the composition comprises about 1 mM to about 50 mM buffer, e.g., about 1 mM to about 40 mM buffer or about 1 mM to about 30 mM. In various aspects, the composition comprises about 5 mM to about 40 mM, optionally, about 10 mM to about 30 mM buffer, about 15 mM to about 25 mM, about 10 mM to about 40 mM buffer, about 15 mM to about 40 mM buffer, or about 20 mM to about 40 mM buffer. In exemplary instances, the DF buffer used to prepare the liquid compositions of the present disclosure comprises about 1 mM to about 50 mM buffer, e.g., about 1 mM to about 40 mM buffer or about 1 mM to about 30 mM. In various aspects, the DF buffer comprises about 5 mM to about 40 mM, optionally, about 10 mM to about 30 mM buffer, about 10 mM to about 40 mM buffer, about 15 mM to about 40 mM buffer, or about 20 mM to about 40 mM buffer. In certain embodiments, the buffer is an acetate buffer. Optionally, the DF buffer comprises about 18 mM to about 22 mM buffer, e.g., about 20 mM buffer. In exemplary alternative aspects, the DF buffer comprises a lower amount of acetate at a lower pH. For instance, such DF buffers may comprise about 8 mM to about 17 mM buffer, e.g., about 10 mM buffer to about 15 mM, about 10 mM, with a pH of about 3.5. Optionally, the buffer is made from glacial acetic acid where sodium hydroxide is added until the target pH is reached. In various instances, the composition comprises about 30 mM to about 38 mM buffer, optionally, about 33 mM to about 38 mM or about 34 mM to about 38 mM buffer (e.g., acetate). In various aspects, the composition comprises about 32 mM to about 36 mM buffer (e.g., acetate). In various aspects, the composition comprises about 34 mM to about 36 mM buffer (e.g., acetate). As described herein, in various aspects, the concentration of the buffer depends on the concentration of the antibody. In various aspects, the liquid composition comprises about 20 mM to about 40 mM, optionally, about 30 mM to about 38 mM, when the concentration of the antibody is about 150 mg/mL. Optionally, when the concentration of the antibody is about 150 mg/mL and the pH of the liquid composition is 4.7-5.3, the composition comprises about 32 mM to about 36 mM or about 33 mM to about 35 mM buffer (e.g., acetate).

In various aspects, the liquid composition comprises, per mL of the liquid composition, (i) about 135 mg to about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, (ii) about 23 mg to about 30 mg proline, (iii) about 0.5 mg to about 2 mg acetate, and (iv) about 0.01% (w/v) polysorbate 80 (PS80), wherein the liquid composition has a pH of about 4.5 to about 5.5. In various aspects, the liquid composition comprises, per mL of the liquid composition, (i) about 150 mg ordesekimab, (ii) about 23 mg to about 30 mg proline, (iii) about 0.5 mg to about 2 mg acetate, and (iv) about 0.01% (w/v) polysorbate 80 (PS80), wherein the liquid composition has a pH of about 4.5 to about 5.5. In various instances, the liquid composition comprises, per mL of the liquid composition, (i) about 150 mg ordesekimab, (ii) about 26.5 mg proline, (iii) about 1.2 mg acetate, and (iv) about 0.01 mg PS80, wherein the liquid composition has a pH of about 5.0.

### Surfactant

The compositions of the present disclosure in various aspects comprise a surfactant. Surfactants are surface active agents that are amphipathic (having a polar head and hydrophobic tail). Surfactants preferentially accumulate at interfaces, resulting in reduced interfacial tension. Use of a surfactant can also help to mitigate formation of large proteinaceous particles. In some aspects, the surfactant present in the compositions of the present disclosure is an amphipathic and/or nonionic surfactant. Exemplary surfactants include polyoxyethylene sorbitan fatty acid esters (e.g. polysorbate 20, polysorbate 80), alkylaryl polyethers, e.g. oxyethylated alkyl phenol (e.g. Triton™ X-100), and poloxamers (e.g. Pluronics®, e.g. Pluronic® F68), and combinations of any of the foregoing, either within a class of surfactants or among classes of surfactants. Polysorbate 20 and polysorbate 80 (and optionally mixtures thereof) are particularly contemplated. The surfactant in exemplary instances is present in the composition at a concentration of about 0.0005% (w/v) to about 0.5% (w/v). In various instances, the surfactant has a final concentration in the liquid composition of about 0.0006% (w/v) to about 0.5% (w/v), 0.0007% (w/v) to about 0.5% (w/v), 0.0008% (w/v) to about 0.5% (w/v), 0.0009% (w/v) to about 0.5% (w/v), 0.001% (w/v) to about 0.5% (w/v), 0.002% (w/v) to about 0.5% (w/v), 0.003% (w/v) to about 0.5% (w/v), 0.004% (w/v) to about 0.5% (w/v), 0.005% (w/v) to about 0.5% (w/v), 0.006% (w/v) to about 0.5% (w/v), 0.007% (w/v) to about 0.5% (w/v), 0.008% (w/v) to about 0.5% (w/v), 0.009% (w/v) to about 0.5% (w/v), 0.01% (w/v) to about 0.5% (w/v), 0.02% (w/v) to about 0.5% (w/v), 0.03% (w/v) to about 0.5% (w/v), 0.04% (w/v) to about 0.5% (w/v), 0.05% (w/v) to about 0.5% (w/v), 0.06% (w/v) to about 0.5% (w/v), 0.07% (w/v) to about 0.5% (w/v), 0.08% (w/v) to about 0.5% (w/v), 0.09% (w/v) to about 0.5% (w/v), 0.1% (w/v) to about 0.5% (w/v), 0.2% (w/v) to about 0.5% (w/v), 0.3% (w/w) to about 0.5% (w/w), 0.4% (w/v) to about 0.5% (w/v), about 0.0005% (w/v) to about 0.4% (w/v), about 0.0005% (w/v) to about 0.3% (w/v), about 0.0005% (w/v) to about 0.2% (w/v), about 0.0005% (w/v) to about 0.1% (w/v), about 0.0005% (w/v) to about 0.09% (w/v), about 0.0005% (w/v) to about 0.08% (w/v), about 0.0005% (w/v) to about 0.07% (w/v), about 0.0005% (w/v) to about 0.06% (w/v), about 0.0005% (w/v) to about 0.05% (w/v), about 0.0005% (w/v) to about 0.04% (w/v), about 0.0005% (w/v) to about 0.03% (w/v), about 0.0005% (w/v) to about 0.02% (w/v), about 0.0005% (w/v) to about 0.01% (w/v), about 0.0005% (w/v) to about 0.009% (w/v), about 0.0005% (w/v) to about 0.008% (w/v), about 0.0005% (w/v) to about 0.007% (w/v), about 0.0005% (w/v) to about 0.006% (w/v), about 0.0005% (w/v) to about 0.005% (w/v), about 0.0005% (w/v) to about 0.004% (w/v), about 0.0005% (w/v) to about 0.003% (w/v), about 0.0005% (w/v) to about 0.002% (w/v), about 0.0005% (w/v) to about 0.001% (w/v), about 0.0005% (w/v) to about 0.0009% (w/v), about 0.0005% (w/v) to about 0.0008% (w/v), about 0.0005% (w/v) to about 0.0007% (w/v), or about 0.0005%((w/v) to about 0.0005% (w/v). In exemplary instances, the surfactant is present at a concentration of about 0.001% (w/v) to about 0.050% (w/v), about 0.005% (w/v) to about 0.025% (w/v) or about 0.01% (w/v)±0.001% (w/v). For instance, the formulation may comprise about 0.005% (w/v) to about 0.05% (w/v) surfactant. In various aspects, the surfactant is a polysorbate, e.g., polysorbate 20 or polysorbate 80 or a mixture thereof. Optionally, the surfactant is present at a concentration less than or about 0.005% (w/v) to about 0.015% (w/v), optionally, about 0.010% (w/v)±0.0025% (w/v) surfactant, or about 0.005% (w/v), 0.010% (w/v), or 0.015% (w/v) surfactant.

### pH, Viscosity, and Osmolality

In various aspects, the liquid composition has a pH which is less than about 7.0, optionally, less than about 6.5, or less than about 6.0. In exemplary aspects, the pH is about 4.50 to about 5.75, e.g., about 4.55 to about 5.75, about 4.60 to about 5.75, about 4.65 to about 5.75, about 4.70 to about 5.75, about 4.75 to about 5.75, about 4.80 to about 5.75, about 4.85 to about 5.75, about 4.90 to about 5.75, about 4.95 to about 5.75, about 5.00 to about 5.75, about 5.05 to about 5.75, about 5.10 to about 5.75, about 5.15 to about 5.75, about 5.20 to about 5.75, about 5.25 to about 5.75, about 5.30 to about 5.75, about 5.35 to about 5.75, about 5.40 to about 5.75, about 5.45 to about 5.75, about 5.50 to about 5.75, about 5.55 to about 5.75, about 5.60 to about 5.75, about 5.65 to about 5.75, about 5.70 to about 5.75, about 4.50 to about 5.70, about 4.50 to about 5.65, about 4.50 to about 5.60, about 4.50 to about 5.55, about 4.50 to about 5.50, about 4.50 to about 5.45, about 4.50 to about 5.40, about 4.50 to about 5.35, about 4.50 to about 5.30, about 4.50 to about 5.25, about 4.50 to about 5.20, about 4.50 to about 5.15, about 4.50 to about 5.10, about 4.50 to about 5.05, about 4.50 to about 5.00, about 4.50 to about 4.95, about 4.50 to about 4.90, about 4.50 to about 4.85, about 4.50 to about 4.80, about 4.50 to about 4.75, about 4.50 to about 4.70, about 4.50 to about 4.65, about 4.50 to about 4.60, or about 4.50 to about 4.55. In various instances, the pH is about 4.7. In various aspects, the pH is about 4.7 to about 5.3, optionally, about 5.0.

In exemplary aspects, the liquid composition is characterized by a reduced viscosity, relative to a liquid composition not comprising arginine glutamate or proline (e.g., compared to a liquid composition comprising 5%-10% (w/v) sucrose). In certain aspects, the composition is characterized by a viscosity of about 5 cP to about 30 cP, e.g., about 5 cP to about 25 cP, about 5 cP to about 20 cP, about 5 cP to about 15 cP, about 5 cP to about 10 cP, about 10 cP to about 25 cP, about 15 cP to about 20 cP, or about 5 cP, about 6 cP, about 7 cP, about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, at a temperature of about 5° C. to about 30° C., when the concentration of the antibody is 100 mg/ml to about 180 mg/mL (e.g., about 120 mg/mL, about 130 mg/mL, about 140 mg/mL, about 150 mg/ml, about 165 mg/mL, about 180 mg/mL). In exemplary aspects, when the concentration of the antibody is about 100 mg/mL to about 165 mg/mL, the composition has a viscosity that is less than about 10 cP at about 25° C. or less than about 20 cP at 5° C. In exemplary aspects, when the concentration of the antibody is greater than about 165 mg/mL, e.g., about 190 mg/mL, the composition has a viscosity that is less than about 30 cP at about 5° C. or less than about 15 cP at 25° C. Unless noted otherwise, all viscosities disclosed herein refers to a viscosity measured using a viscometer at 25° C. and at a shear rate of about 1000 s⁻¹.

In exemplary aspects, the liquid composition is intended for administration to a subject by injection or infusion, and thus the composition is isotonic with the intended site of administration. Accordingly, in various instances of the present disclosure, the liquid composition is an isotonic liquid composition. In exemplary instances, the osmolality of the composition is in a range of about 250 mOsm/kg to about 350 mOsm/kg, about 270 to about 350 mOsm/kg, or about 285 to about 345 mOsm/kg, or about 300 to about 315 mOsm/kg. Herein, the osmolality unit “mOsm/kg” is synonymous with “mOsmol/kg”. For example, if the solution is in a form intended for administration parenterally, it can be isotonic with blood (about 300 mOsm/kg osmolality). In exemplary aspects, the liquid pharmaceutical formulation has an osmolality in less than about 450 mOsm/kg (e.g., less than about 400 mOsm/kg).

### Stability

The presently disclosed liquid compositions demonstrate high stability as evidenced by the low % initial HMW species as determined by Size Exclusion Chromatography (SEC). As used herein, “initial HMW species” refers to the HMW species formed upon combining the antibody with the components of the liquid composition and/or shortly after the components are combined with the antibody. Initial HMW species refers to the HMW species initially formed and/or wherein the storage time is zero. In exemplary aspects, less than 5% (e.g., less than or about 4%, less than or about 3%, less than or about 2%, less than or about 1%) initial HMW species is detected by SEC.

In various instances, the presently disclosed liquid compositions are storage-stabilized (or storage-stable) as demonstrated by the reduced amounts of aggregates and/or reduced aggregate formation rates following storage. As described herein, the stability of such liquid compositions is shown by the reduced amounts of HMWS and/or reduced HMWS formation rates following storage for varied time periods and at varied temperatures. In general, higher stability liquid compositions are associated with lower amounts of HMWS, lower HMWS formation rates, and/or higher antibody main peaks at higher storage temperatures, relative to lower temperatures. As used herein, the term “high molecular weight species” or “HMWS” refers to higher order aggregates of the antibody of the formulations, as well as lower order aggregates of the antibody of the formulations. Lower-order aggregates, include, for example, dimer species. The aggregate amounts and rates of formation may be measured or monitored by techniques, such as, e.g., SE-UHPLC. SE-UHPLC chromatograms of the antibody, in some instances, show peaks around 2.2 to 2.8 minutes representing the amount of HMWS of the liquid composition, and a peak around 3 minutes reflecting the amount of intact, non-aggregated forms of the antibody. Relative to storage at 4° C., storage at 37° C. allows for the acceleration of a stability assay such that the stability of a particular formulation may be determined in a shorter period of time, relative to the storage time period at 4° C. For example, storage at 37° C. for 1, 2, or 3 months may be indicative or predictive of storage at 4° C. for 36 months.

In one type of embodiment, a storage-stable liquid composition as described herein will show a reduced extent and rate of formation of HMWS following 3 months of storage at 37° C., as compared to an equivalent-concentration control formulation consisting of acetate and sucrose as excipients.

In another type of embodiment, a storage-stable liquid composition as described herein and including an arginine glutamate or proline will show a reduced extent of formation of HMWS following 1 month of storage at 37° C., as compared to an equivalent control formulation without the arginine glutamate or proline. For example, the extent of formation can be reduced such that the % amount of HMWS by SE-UPHLC is lower by at least about 0.1%, or about 0.2%, or about 0.3%, or about 0.4%, or about 0.5%, or about 0.6%, or about 0.7%, for example in a range of about 0.1% to about 2%, or about 0.1% to about 1%, compared to the control formulation following 1 month of storage at 37° C.

In another type of embodiment a storage-stable liquid composition as described herein will have a low amount of HMWS following 1 month storage at 37° C., by SE-UHPLC. For example, the amount of HMWS can be not more than 2%, or less than 2%, or not more than 1.9%, or less than 1.9%, or not more than 1.8%, or less than 1.8%, or not more than 1.7%, or less than 1.7%, or not more than 1.6%, or less than 1.6%, or not more than 1.5%, or less than 1.5%, or not more than 1.4%, or less than 1.4%, or not more than 1.3%, or less than 1.3%, or not more than 1.2%, or less than 1.2%. In other examples the amount of HMWS can be, for example in a range of about 0.01% to about 2%, or about 0.01% to about 1.9%, or about 0.01% to about 1.8%, or about 0.01% to about 1.7%, or about 0.01% to about 1.6%, or about 0.01% to about 1.5%, or about 0.01% to about 1.4%, or about 0.01% to about 1.3%, or about 0.01% to about 1.2%. In another type of embodiment, the amount of HMWS following 1 month storage at 37° C. by SE-UHPLC can be greater than 2%, e.g. greater than 2% and up to 3%, while the reduced rate of aggregation provided by the amino acid aggregation inhibitor will allow for a suitable product shelf life, e.g. up to three years, or up to two years.

In another type of embodiment a storage-stable liquid composition as described herein will have a low amount of HMWS following 3 months storage at 37° C., by SE-UHPLC. For example, the amount of HMWS can be not more than 2%, or less than 2%, or not more than 1.9%, or less than 1.9%, or not more than 1.8%, or less than 1.8%, or not more than 1.7%, or less than 1.7%, or not more than 1.6%, or less than 1.6%, or not more than 1.5%, or less than 1.5%, or not more than 1.4%, or less than 1.40%, or not more than 1.3%, or less than 1.3%, or not more than 1.2%, or less than 1.2%. In other examples the amount of HMWS can be, for example in a range of about 0.01% to about 2%, or about 0.01% to about 1.9%, or about 0.01% to about 1.8%, or about 0.01% to about 1.7%, or about 0.01% to about 1.6%, or about 0.01% to about 1.5%, or about 0.01% to about 1.4%, or about 0.01% to about 1.3%, or about 0.01% to about 1.2%.

In another type of embodiment a storage-stable liquid composition as described herein will have a low amount of HMWS following 36 months storage at 4° C., by SE-UHPLC. For example, the amount of HMWS can be not mom than 2%, or less than 2%, or not more than 1.9%, or less than 1.9%, or not more than 1.8%, or less than 1.8%, or not more than 1.7%, or less than 1.7%, or not more than 1.6%, or less than 1.6%, or not more than 1.5%, or less than 1.5%, or not more than 1.4%, or less than 1.4%, or not more than 1.3%, or less than 1.3%, or not more than 1.2%, or less than 1.2%. In other examples the amount of HMWS can be, for example in a range of about 0.01% to about 2%, or about 0.01% to about 1.9%, or about 0.01% to about 1.8%, or about 0.01% to about 1.7%, or about 0.01% to about 1.60%, or about 0.01% to about 1.5%, or about 0.01% to about 1.4%, or about 0.01% to about 1.3%, or about 0.01% to about 1.2%.

In another type of embodiment a storage-stable liquid composition as described herein will have a high amount of the antibody main peak following 1 month storage at 37° C. by SE-UHPLC. For example, the amount of the main peak can be at least 95%, or greater than 95%, or at least 96%, or greater than 96%, or at least 97%, or greater than 97%, or at least 97.5%, or greater than 97.5%, or at least 98%, or greater than 98%, or at least 98.1%, or greater than 98.1%, or at least 98.2%, or greater than 98.2%, or at least 98.3%, or greater than 98.3%, or at least 98.4%, or greater than 98.4%, or at least 98.5%, or greater than 98.5%, or at least 98.6%, or greater than 98.6%. In other examples the amount of main peak can be, for example in a range of about 95% to about 99.9%, or about 96% to about 99.9%, or about 97% to about 99.9%, or about 97.5% to about 99.9%, or about 98% to about 99.9%, or about 98.1% to about 99.9%, or about 98.2% to about 99.9%, or about 98.3% to about 99.9%, or about 98.4% to about 99.9%, or about 98.5% to about 99.9%, or about 98.6% to about 99.9%.

In another type of embodiment a storage-stable liquid composition as described herein will have a high amount of the antibody main peak following 3 months storage at 37° C., by SE-UHPLC. For example, the amount of the main peak can be at least 95%, or greater than 95%, or at least 96%, or greater than 96%, or at least 97%, or greater than 97%, or at least 97.5%, or greater than 97.5%, or at least 98%, or greater than 98%, or at least 98.1%, or greater than 98.1%, or at least 98.2%, or greater than 98.2%, or at least 98.3%, or greater than 98.3%, or at least 98.4%, or greater than 98.4%, or at least 98.5%, or greater than 98.5%, or at least 98.6%, or greater than 98.6%. In other examples the amount of main peak can be, for example in a range of about 95% to about 99.9%, or about 96% to about 99.9%, or about 97% to about 99.9%, or about 97.5% to about 99.9%, or about 98% to about 99.9%, or about 98.1% to about 99.9%, or about 98.2% to about 99.9%, or about 98.3% to about 99.9%, or about 98.4% to about 99.9%, or about 98.5% to about 99.9%, or about 98.6% to about 99.9%.

In another type of embodiment a storage-stable liquid composition as described herein will have a high amount of the antibody main peak following 36 months storage at 4° C., by SE-UHPLC. For example, the amount of the main peak can be at least 95%, or greater than 95%, or at least 96%, or greater than 96%, or at least 97%, or greater than 97%, or at least 97.5%, or greater than 97.5%, or at least 98%, or greater than 98%, or at least 98.1%, or greater than 98.1%, or at least 98.2%, or greater than 98.2%, or at least 98.3%, or greater than 98.3%, or at least 98.4%, or greater than 98.4%, or at least 98.5%, or greater than 98.5%, or at least 98.6%, or greater than 98.6%. In other examples the amount of main peak can be, for example in a range of about 95% to about 99.9%, or about 96% to about 99.9%, or about 97% to about 99.9%, or about 97.5% to about 99.9%, or about 98% to about 99.9%, or about 98.1% to about 99.9%, or about 98.2% to about 99.9%, or about 98.3% to about 99.9%, or about 98.4% to about 99.9%, or about 98.5% to about 99.9%, or about 98.6% to about 99.9%.

In further embodiments, it is contemplated that the storage-stable liquid composition will have both a low amount of HMWS and a high amount of main peak, according to a specification described above, following storage.

In exemplary aspects, the storage-stable liquid compositions comprise not more than about 4% high molecular weight species (HMWS) and/or comprise more than about 96% of the antibody main peak, as measured by SE-UHPLC, following storage. In exemplary aspects, the storage-stable liquid compositions comprise not more than about 3% high molecular weight species (HMWS) and/or comprise more than about 97% of the antibody main peak, as measured by SE-UHPLC, following storage. In exemplary aspects, the storage-stable liquid compositions comprise less than about 2% HMWS and/or more than about 98% of the antibody main peak, as measured by SE-UHPLC, following storage. In exemplary aspects, the storage is at a temperature of about 2° C. to about 8° C. (e.g., about 2° C., about 3° C., about 4° C., about 5° C., about 6° C., about 7° C., about 8° C.) for at least 12 months, 24 months, or 36 months (e.g., at least or about 12 months, at least or about 16 months, at least or about 20 months, at least or about 24 months, at least or about 28 months, at least or about 32 months, at least or about 36 months, optionally, longer). In exemplary aspects, the storage is at about 20° C. to about 30° C. (e.g., about 21° C. to about 30° C., about 22° C. to about 30° C., about 23° C. to about 30° C., about 24° C. to about 30° C., about 25° C. to about 30° C., about 26° C. to about 30° C., about 27° C. to about 30° C., about 28° C. to about 30° C. about 28° C. to about 30° C., about 20° C. to about 29° C., about 20° C. to about 28° C., about 20° C. to about 27° C., about 20° C. to about 26° C., about 20° C. to about 25° C., about 20° C. to about 24° C. about 20° C. to about 23° C., about 20° C. to about 22° C.) for about 1 month (e.g., about 26 days, about 27 days, about 28 days, about 29 days, about 30 days, about 31 days, about 32 days, about 33 days, about 34 days, about 35 days, about 36 days). In exemplary aspects, the storage comprises a first storage followed by a second storage and the first storage is at about 2° C. to about 8° C. for at least 12 months, 24 months, or 36 months and the second storage is at about 20° C. to about 30° C. for about 1 month. In exemplary instances, the storage-stable liquid compositions comprise not more than 2% high molecular weight species (HMWS), or less than 2% HMWS, or not more than 1.9% HMWS, or less than 1.9% HMWS, or not more than 1.8% HMWS, or less than 1.8% HMWS, or not more than 1.7% HMWS, or less than 1.7% HMWS, or not more than 1.6% HMWS, or less than 1.6% HMWS, or not more than 1.5% HMWS, or less than 1.5% HMWS, or not more than 1.4% HMWS, or less than 1.4% HMWS, or not more than 1.3% HMWS, or less than 1.3% HMWS, or not more than 1.2% HMWS, or less than 1.2% HMWS. In other examples the amount of HMWS can be, for example in a range of about 0.01% to about 2% HMWS, or about 0.01% to about 1.9% HMWS, or about 0.01% to about 1.8% HMWS, or about 0.01% to about 1.7% HMWS, or about 0.01% to about 1.6% HMWS, or about 0.01% to about 1.5% HMWS, or about 0.01% to about 1.4% HMWS, or about 0.01% to about 1.3% HMWS, or about 0.01% to about 1.2% HMWS, optionally, as measured by SE-UHPLC. In alternative or additional aspects, the storage-stable liquid compositions comprise more than 98% of the antibody main peak, or at least 95% antibody main peak, or greater than 95% antibody main peak, or at least 96% antibody main peak, or greater than 9% antibody main peak, or at least 97% antibody main peak, or greater than 97% antibody main peak, or at least 97.5% antibody main peak, or greater than 97.5% antibody main peak, or at least 98% antibody main peak, or greater than 98% antibody main peak, or at least 98.1% antibody main peak, or greater than 98.1% antibody main peak, or at least 98.2% antibody main peak, or greater than 98.2% antibody main peak, or at least 98.3% antibody main peak, or greater than 98.3% antibody main peak, or at least 98.4% antibody main peak, or greater than 98.4% antibody main peak, or at least 98.5% antibody main peak, or greater than 98.5% antibody main peak, or at least 98.6% antibody main peak, or greater than 98.6% antibody main peak. In other examples the amount of main peak can be, for example in a range of about 95% to about 99.9% antibody main peak, or about 96% to about 99.9% antibody main peak, or about 97% to about 99.9% antibody main peak, or about 97.5% to about 99.9% antibody main peak, or about 98% to about 99.9% antibody main peak, or about 98.1% to about 99.9% antibody main peak, or about 98.2% to about 99.9% antibody main peak, or about 98.3% to about 99.9% antibody main peak, or about 98.4% to about 99.9% antibody main peak, or about 98.5% to about 99.9% antibody main peak, or about 98.6% to about 99.9% antibody main peak, optionally, as measured by SE-UHPLC.

In exemplary aspects, less than 5% (e.g., less than or about 4%, less than or about 3%, less than or about 2%, less than or about 1%) of the antibody is degraded after storage at 2° C. to 8° C. for at least or about 12 months, as determined by SEC. In various aspects, less than 5% (e.g., less than or about 4%, less than or about 3%, less than or about 2%, less than or about 1%) of the antibody is degraded after storage at 2° C. to 8° C. for about 20 months to about 26 months, as determined by SEC. In various instances, less than 5% (e.g., less than or about 4%, less than or about 3%, less than or about 2%, less than or about 1%) of the antibody is degraded after storage at 2° C. to 8° C. for about 30 to about 40 months, as determined by SEC. Optionally, less than 5% (e.g., less than or about 4%, less than or about 3%, less than or about 2%, less than or about 1%) of the antibody is degraded after storage at 2° C. to 8° C. for about 36-40 months, as determined by SEC. In various instances, less than about 5% of the antibody is degraded after storage at about 2° C. to about 8° C. for at least or about 12 months, as determined by SEC. In various aspects, less than about 5% of the antibody is degraded after storage at about 2° C. to about 8° C. for about 20 months to about 26 months, as determined by SEC. In exemplary instances, less than about 5% of the antibody is degraded after storage at about 2° C. to about 8° C. for about 30 to about 40 months, as determined by Size Exclusion Chromatography (SEC). In exemplary instances, less than about 5% of the antibody is degraded after storage at about 2° C. to about 8° C. for about 2 years to about 3 years, as determined by Size Exclusion Chromatography (SEC). Also, for example, less than 5% of the antibody is degraded after about 24 months to about 36 months of storage at 2° C. to 8° C. as determined by Size Exclusion Chromatography (SEC), optionally, wherein less than 2% of the antibody is degraded after 24 months or 36 months of storage at 2° C. to 8° C. In various aspects, less than 5% of the antibody is degraded after at least 2 weeks (optionally, after at least 1 month, after at least 2 months, after at least 3 months, after at least 4 months, after at least 5 months or after at least 6 months) of storage at about room temperature (e.g., 25° C.), as determined by SEC. In various instances, less than 5% of the antibody is degraded after about 24 months to about 36 months of storage at 2° C. to 8° C. followed by at least 2 weeks or at least about 1 month or at least about 2 months of storage at about room temperature (e.g., 25° C.), as determined by SEC. Optionally, less than about 5% of the antibody is degraded after storage at a temperature greater than about 20° C. for at least or about 2 weeks, as determined by Size Exclusion Chromatography (SEC), optionally, for at least or about 4 weeks or about 8 weeks. In various aspects, the temperature is greater than or about 25° C. or greater than or about 30° C. or greater than or about 40° C.

In exemplary aspects, less than 5% (e.g., less than or about 4%, less than or about 3%, less than or about 2%, less than or about 1%) HMW species is detected by SEC, after storage at 2° C. to 8° C. for at least or about 12 months. In various aspects, less than 5% (e.g., less than or about 4%, less than or about 3%, less than or about 2%, less than or about 1%) HMW species is detected by SEC, after storage at 2° C. to 8° C. for about 20 months to about 26 months. In various instances, less than 5% (e.g., less than or about 4%, less than or about 3%, less than or about 2%, less than or about 1%) HMW species is detected by SEC after storage at 2° C. to 8° C. for about 30 to about 40 months. Optionally, less than 5% (e.g., less than or about 4%, less than or about 3%, less than or about 2%, less than or about 1%) HMW species is detected by SEC after storage at 2° C. to 8° C. for about 36-40 months.

In exemplary aspects, the presently disclosed liquid compositions demonstrate stability against one or more stresses, including, for example, stress caused by shipping, exposure to light, exposure to heat, exposure to air, and/or a freeze-thaw cycle (e.g., the stress caused by thawing a liquid composition that was stored at a frozen temperature). In various instances, the liquid composition is stable against a freeze-thaw, as evidenced by the low amount of precipitates of the components of the liquid composition in the liquid composition after a freeze thaw.

### Articles of Manufacture, Syringes, and Vials

An article of manufacture is further provided herein. In exemplary embodiments, the article comprises the composition of the present disclosure, optionally, comprising about 1 mL to about 5 mL, e.g., about 1 mL to about 3 mL of the liquid composition. In exemplary aspects of the disclosure, the composition is provided for storage or use, e.g. in a single-use vial, single-use syringe, or glass, glass-lined, glass-coated primary container or auto-injector. In exemplary aspects, the composition is provided in a single use system bag or a polycarbonate carboy for frozen storage. In alternative aspects, the composition is contained in glass vials or syringes for storage at 2° C. to 8° C. A pre-filled syringe comprising the presently disclosed composition, optionally, comprising about 1 mL to about 5 mL, e.g., about 1 mL to about 3 mL of the composition, is additionally provided herein. Further provided is a vial comprising the presently disclosed composition, optionally, comprising about 1 mL to about 5 mL, e.g., about 1 mL to about 3 mL of the liquid composition. In various aspects, the article, prefilled syringe, or vial comprises about 2 mL to about 3 mL (e.g., about 2.1 mL, about 2.2 mL, about 2.3 mL, about 2.4 mL, about 2.5 mL, about 2.6 mL, about 2.7 mL, about 2.8 mL, about 2.9 mL) of the composition of the present disclosure and in various aspects, the composition comprises antibody at a concentration of about 150 mg/mL.

In exemplary instances, the composition is provided for use in a delivery system which is off-the-shelf and/or designed for self-administration. In exemplary aspects, the composition is provided in a pre-filled syringe or an autoinjector, a pen injector, a dual-chamber pen, and the like. Such products are known in the art and are commercially available.

The composition of the present disclosure can be suitable for administration by any acceptable route, including parenteral, and specifically injection using a syringe. For example, the injection can be to the upper arm, upper thigh, or abdomen. Other routes include, for example, subcutaneous, intravenous, intradermal, intramuscular, intraperitoneal, internodal and intrasplenic.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored in a ready-to-use form.

### Methods of Preparing Liquid Compositions

Methods of preparing a liquid composition comprising a target concentration of a monoclonal antibody are further provided by the present disclosure. In exemplary embodiments, the target concentration is greater than about 100 mg/mL. In various aspects, the target concentration is any of those described herein, including, but not limited to those described in the Antibody Concentration section herein. Accordingly, in various aspects, the target concentration is about 110 mg/mL to about 300 mg/mL, e.g., about 110 mg/mL to about 290 mg/mL, about 110 mg/mL to about 280 mg/mL, about 110 mg/mL to about 270 mg/mL, about 110 mg/mL to about 260 mg/mL, about 110 mg/mL to about 250 mg/mL, about 110 mg/mL to about 240 mg/mL, about 110 mg/mL to about 230 mg/mL, about 110 mg/mL to about 220 mg/mL, about 110 mg/mL to about 210 mg/mL, about 110 mg/mL to about 200 mg/mL, about 110 mg/mL to about 190 mg/mL, about 110 mg/mL to about 180 mg/mL, about 110 mg/mL to about 170 mg/mL, about 110 mg/mL to about 160 mg/mL, about 110 mg/mL to about 150 mg/mL, about 110 mg/mL to about 140 mg/mL, about 110 mg/mL to about 130 mg/mL, about 110 mg/mL to about 120 mg/mL, about 120 mg/mL to about 300 mg/mL, about 130 mg/mL to about 300 mg/mL, about 140 mg/mL to about 300 mg/mL, about 150 mg/mL to about 300 mg/mL, about 160 mg/mL to about 300 mg/mL, about 170 mg/mL to about 300 mg/mL, about 180 mg/mL to about 300 mg/mL, about 190 mg/mL to about 3M00 mg/mL, about 200 mg/mL to about 300 mg/mL, about 210 mg/mL to about 300 mg/mL, about 220 mg/mL to about 300 mg/mL, about 230 mg/mL to about 300 mg/mL, about 240 mg/mL to about 3M00 mg/mL, about 250 mg/mL to about 300 mg/mL, about 260 mg/mL to about 300 mg/mL, about 270 mg/mL to about 300 mg/mL, about 280 mg/mL to about 300 mg/mL, or about 290 mg/mL to about 300 mg/mL. In various instances, the target concentration of the antibody is about 120 mg/mL to about 250 mg/mL, optionally, about 120 mg/mL to about 240 mg/mL, about 120 mg/mL to about 230 mg/mL, about 120 mg/mL to about 220 mg/mL, about 120 mg/ml to about 210 mg/mL, about 120 mg/mL to about 200 mg/mL, about 120 mg/mL to about 190 mg/mL, about 120 mg/mL to about 180 mg/mL, about 120 mg/mL to about 170 mg/mL, about 120 mg/ml to about 160 mg/mL, about 120 mg/mL to about 150 mg/mL, about 120 mg/mL to about 140 mg/mL, about 120 mg/mL to about 130 mg/mL, about 130 mg/mL to about 250 mg/mL, about 140 mg/mL to about 250 mg/mL, about 150 mg/mL to about 250 mg/mL, about 160 mg/mL to about 250 mg/mL, about 170 mg/mL to about 250 mg/mL, about 180 mg/mL to about 250 mg/mL, about 190 mg/mL to about 250 mg/mL, about 200 mg/mL to about 250 mg/mL, about 210 mg/mL to about 250 mg/mL, about 220 mg/mL to about 250 mg/mL, about 230 mg/mL to about 250 mg/mL, or about 240 mg/mL to about 250 mg/mL. In various instances, the target concentration of the antibody is about 160 mg/mL to about 250 mg/mL, e.g., about 180 mg/mL to about 225 mg/mL, or about 180 mg/mL to about 200 mg/mL. In some aspects, the target concentration of about 130 mg/mL to about 225 mg/mL, about 130 mg/mL to about 220 mg/mL or about 130 mg/mL to about 200 mg/mL. In various instances, the target concentration of about 135 mg/mL to about 165 mg/mL, optionally about 140 mg/mL to about 160 mg/mL. In exemplary aspects, the target concentration is about 120 mg/mL antibody, about 140 mg/mL, about 150 mg/mL or about 165 mg/mL

In exemplary embodiments, the method comprises formulating the antibody with a diafiltration (DF) buffer comprising (i) arginine glutamate or (ii) proline and a buffer. The arginine glutamate in exemplary aspects comprises L-arginine base and L-glutamic acid. In various instances, the antibody is present in a first liquid composition and a buffer exchange is carried out to place the antibody in the DF buffer. Following the buffer exchange, a surfactant is added. Optionally, the method comprises adjusting the pH to a target pH. In various aspects, no adjustment of the pH is needed following the buffer exchange. In particular aspects, when the DF buffer comprises arginine glutamate, adjustment of the pH is not needed.

In various aspects, the method comprises (a) combining the monoclonal antibody with a diafiltration (DF) buffer comprising (i) arginine base and (ii) glutamate, wherein the molar ratio of arginine to glutamate is about 0.7:1.0 to about 1.1:1.0, and (b) adding a surfactant. In various aspects, the molar ratio of arginine to glutamate is about 0.8:1.0 to about 1.1:1.0. In various aspects, the DF buffer comprises about 50 mM to about 300 mM arginine base. Optionally, the DF buffer comprises about 85 mM to about 190 mM arginine or about 135 mM to about 165 mM arginine. In various instances, the DF buffer comprises about 155 mM to about 185 mM glutamate. Optionally, the DF buffer comprises about 150 mM arginine and about 170 mM glutamate. Optionally, the DF buffer comprises about 136 mM arginine and about 159 mM glutamate. In various instances, the DF buffer comprises about 85 mM to about 190 mM arginine and about 85 mM to about 200 mM glutamate. In various instances, the DF buffer is made with about 80 mM to about 240 mM arginine base and about 80 mM to about 240 mM glutamic acid. In various aspects, the DF buffer comprises about 100 mM to about 180 mM arginine (e.g., about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 180 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 135 mM to about 155 mM) and about 110 mM to about 240 mM glutamate (e.g., about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 140 mM to about 160 mM, about 145 mM to about 155 mM glutamate). In various instances, the DF buffer is made with about 100 mM to about 170 mM arginine base. In various instances, the DF buffer comprises about 120 mM to about 150 mM arginine base. In various instances, the DF buffer comprises about 136 mM arginine base. In various instances, the DF buffer is made with about 120 mM to about 200 mM glutamic acid. In various instances, the DF buffer is made with about 140 mM to about 175 mM glutamic acid. In various instances, the DF buffer is made with about 159 mM glutamic acid. In various instances, the DF buffer is made with about 136 mM arginine base and about 159 mM glutamic acid. In various instances, the DF buffer comprises about 85 mM to about 125 mM arginine and about 85 mM to about 225 mM glutamate. In various instances, the DF buffer comprises about 135 mM to about 165 mM arginine base and about 155 mM to about 185 mM glutamate or about 135 mM to about 165 mM arginine base and about 155 mM to about 185 mM glutamate. In various instances, the DF buffer comprises about 135 mM to about 145 mM arginine and about 145 mM to about 155 mM glutamate. In various aspects, the DF buffer comprise about 150 mM arginine base and about 170 mM glutamate. In exemplary aspects, the pH of the DF buffer is about the same as the final pH (e.g., the target pH) of the prepared liquid composition. Optionally, the final pH of the prepared liquid composition is about 4.5 to about 6.5. In exemplary instances, the first liquid composition comprises the monoclonal antibody at a concentration greater than the target concentration and the method further comprises diluting the monoclonal antibody with DF buffer to attain the target concentration, prior to adding the surfactant. The surfactant may be any surfactant known in the art or described herein. Preferably, the surfactant is polysorbate 80, and optionally, the method comprises adding an amount of PS80 to attain a final concentration of about 0.01% (w/v).

In alternative aspects, the method comprises (a) combining the monoclonal antibody with a DF buffer comprising about 150 mM to about 300 mM proline and about 10 mM to about 50 mM acetate, and (b) adding a surfactant. In exemplary aspects, the DF buffer comprises proline in a range of about 175 mM to about 375 mM proline or about 200 mM to about 350 mM proline. Optionally, the DF buffer comprises proline at a concentration of about 200 mM to about 325 mM, about 200 mM to about 300 mM, about 200 mM to about 275 mM, about 200 mM to about 250 mM, about 200 mM to about 225 mM, about 225 mM to about 350 mM, about 250 mM to about 350 mM, about 275 mM to about 350 mM, about 300 mM to about 350 mM, or about 325 mM to about 350 mM. In various instances, the proline concentration in the DF buffer is about 200 mM to about 300 mM, about 225 mM to about 275 mM, about 235 mM to about 265 mM, or about 240 mM to about 260 mM. In various instances, the DF buffer comprises about 1 mM to about 50 mM acetate, e.g., about 1 mM to about 40 mM acetate or about 1 mM to about 30 mM acetate. In various aspects, the DF buffer comprises about 5 mM to about 40 mM, about 10 mM to about 30 mM acetate, optionally, about 15 mM to about 30 mM acetate, about 20 mM to about 30 mM acetate, or about 10 mM to about 25 mM acetate. In exemplary aspects, the acetate is present in the DF buffer at a concentration of about 10 mM, about 11 mM, about 12 mM, about 13 mM, about 14 mM, about 15 mM, about 16 mM, about 17 mM, about 18 mM, about 19 mM, about 20 mM, about 21 mM, about 22 mM, about 23 mM, about 24 mM, about 25 mM, about 26 mM, about 27 mM, about 28 mM, about 29 mM or about 30 mM.

The liquid compositions prepared by the presently disclosed methods are furthermore provided herein.

### Treatment

Methods of treating a disease are provided herein. The methods of treating, in exemplary embodiments, comprise administered to a subject with the disease a liquid composition of the present disclosure in an amount effective to treat the disease. As used herein, the term “treat,” as well as words related thereto, do not necessarily imply 100% or complete treatment. Rather, there are varying degrees of treatment of which one of ordinary skill in the art recognizes as having a potential benefit or therapeutic effect. In this respect, the methods of treating a disease of the present disclosure can provide any amount or any level of treatment. Furthermore, the treatment provided by the methods of the present disclosure can include treatment of one or more conditions or symptoms or signs of the disease being treated. Also, the treatment provided by the methods of the present disclosure can encompass slowing the progression of the disease. For example, when the disease is cancer, the methods can treat disease by virtue of enhancing the T cell activity or NK cell activity or an immune response against the cancer, reducing tumor or cancer growth, reducing metastasis of tumor cells, increasing cell death of tumor or cancer cells, and the like. In various aspects, the methods treat by way of delaying the onset or recurrence of the disease. In various aspects, the methods treat by way increasing the survival of the subject. In exemplary instances, the onset or recurrence or the occurrence is delayed by at least 1 day, 2 days, 4 days, 6 days, 8 days, 10 days, 15 days, 30 days, two months, 3 months, 4 months, 6 months, 1 year, 2 years, 3 years, 4 years, or more. Accordingly, the present disclosure further provides methods of delaying the onset or recurrence of the disease or increasing the survival of the subject, comprising administering to the subject the isotonic liquid composition of the present disclosure.

### Disease

In various embodiments, the disease is celiac disease, which results in debilitating symptoms, including gut mucosal damage and potentially serious medical complications.

Celiac disease is a systemic autoimmune disease triggered by gluten consumption in genetically susceptible individuals (Green and Cellier, 2007). Currently ˜1% of the United States (US) and European Union (EU) populations are affected by celiac disease, albeit only 10-20% of celiac patients are diagnosed. Celiac was the first autoimmune disease with an identified antigen; gluten, the main protein present in some of the most common cereals (e.g., wheat, barley, rye). Modern diets are increasingly enriched with gluten and it is also used as an additive in processed foods, cosmetics, and oral medications. Gluten is the second most common food ingredient after sugar and, in some countries, is present in up to 80% of foodstuff. The ubiquitous presence of gluten makes total avoidance very difficult, if not impossible. As little as 50 mg/day (a normal diet contains greater than 10 g/day) triggers activation of T cells in the small bowel and causes intestinal mucosal damage (Catassi et al., 2007). For this reason, more than 50% of celiac patients on a glutted-free diet (GFD) continue to present with active disease and intestinal immune activation and mucosal atrophy (Lee et al., 2003; Cranney et al., 2007; Hopper et al., 2007; Midhagen et al., 2003). Patients who continue to have symptoms despite attempting to maintain a GFD are deemed to have non-responsive celiac disease (NRCD). NRCD has been defined as “persistent symptoms, signs or laboratory abnormalities typical of celiac disease, despite 6-12 months of dietary gluten avoidance” (Rubio-Tapia et al., 2013). Patient support groups and experts agree that alternative treatment options independent of, or in combination with, GFD are urgently needed to improve the quality of life for celiac patients.

A rare but specific complication of persistent exposure to gluten in celiac disease is the development of refractory celiac disease (RCD), which affects approximately 1% of celiac patients (Lebwohl et al., 2013). RCD is characterized by severe intestinal mucosal atrophy and gastrointestinal symptoms in the absence of gluten consumption and in the presence of small bowel aberrant IELs (Verbeek et al., 2008, vanWanrooij et al., 2014). RCD patients can be further classified according to the proportion and characteristics of aberrant IELs. Patients with a low proportion of aberrant IELs, defined as less than 20% of total IELs (less than 20 IELs per 100 epithelial cells), as determined by flow cytometry, are referred to as Type 1 RCD (RCD-1). These aberrant IELs are generally polyclonal, and RCD-I patients are not at increased risk of developing overt extra epithelial lymphoma (i.e., enteropathy-associated T cell lymphoma [EATL]) and have a typical 5-year survival (vanWanrooij et al., 2014). To treat RCD-I, corticosteroids (local or systemic), azathioprine, purinethol, anti-TNF agents, or cladribine may be used (Brar et al., 2007; Goerres et al., 2003) with clinical and histological improvement. FIG. 3 illustrates the pathophysiology of celiac and refractory celiac disease, as described by Schuppan et al. When the proportion of aberrant IELs reaches or exceeds 20%, patients are diagnosed with Type II RCD (RCD-II). In RCD-II, the IELs are typically monoclonal and the risk of developing EATL is dramatically increased to greater than 50% (Nijeboer et al., 2015). The aberrant IELs proliferate in the absence of gluten due to accumulation of anti-apoptotic mechanisms, hence the term “refractory” indicating that the disease, a non-Hodgkin slow-growing intraepithelial lymphoma, appears not to be dependent on gluten since it is not responsive to the strictest GFD.

Thus, in various embodiments, the disease is non-responsive celiac disease or refractory celiac disease. In other embodiments, the disease is enteropathy-associated T-cell lymphoma, or non-celiac gluten sensitivity. Each of the forgoing are described in International Patent Publication No. WO 2017/217985, the entire contents of which are incorporated herein by reference.

In various embodiments, the disease is a cancer or solid tumor. The cancer treatable by the methods disclosed herein can be any cancer, e.g., any malignant growth or tumor caused by abnormal and uncontrolled cell division that may spread to other parts of the body through the lymphatic system or the blood stream. The cancer in some aspects is one selected from the group consisting of acute lymphocytic cancer, acute myeloid leukemia, alveolar rhabdomyosarcoma, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, gastrointestinal carcinoid tumor, Hodgkin lymphoma, hypopharynx cancer, kidney cancer, larynx cancer, liver cancer, lung cancer, malignant mesothelioma, melanoma, multiple myeloma, nasopharynx cancer, non-Hodgkin lymphoma, ovarian cancer, pancreatic cancer, peritoneum, omentum, and mesentery cancer, pharynx cancer, prostate cancer, rectal cancer, renal cancer (e.g., renal cell carcinoma (RCC)), small intestine cancer, soft tissue cancer, stomach cancer, testicular cancer, thyroid cancer, ureter cancer, and urinary bladder cancer. In particular aspects, the cancer is selected from the group consisting of: head and neck, ovarian, cervical, bladder and oesophageal cancers, pancreatic, gastrointestinal cancer, gastric, breast, endometrial and colorectal cancers, hepatocellular carcinoma, glioblastoma, bladder, lung cancer, e.g., non-small cell lung cancer (NSCLC), bronchioloalveolar carcinoma. In particular embodiments, the tumor is non-small cell lung cancer (NSCLC), head and neck cancer, renal cancer, triple negative breast cancer, and gastric cancer. In exemplary aspects, the subject has a tumor (e.g., a solid tumor, a hematological malignancy, or a lymphoid malignancy) and the pharmaceutical composition is administered to the subject in an amount effective to treat the tumor in the subject. In other exemplary aspects, the tumor is non-small cell lung cancer (NSCLC), small cell lung cancer (SCLC), head and neck cancer, renal cancer, breast cancer, melanoma, ovarian cancer, liver cancer, pancreatic cancer, colon cancer, prostate cancer, gastric cancer, lymphoma or leukemia, and the pharmaceutical composition is administered to the subject in an amount effective to treat the tumor in the subject. In various instances, the disease is one described in International Patent Publication No. WO 2019/140196 or WO 2015/031667, the entire contents of each are incorporated herein by reference.

In exemplary instances, the presently disclosed method of treating a disease encompasses treatment or prevention of a skeletal-related event (SRE), treatment or prevention of a giant cell tumor of bone, treatment or prevention of hypercalcemia of malignancy, treatment or prevention of osteoporosis, or increasing bone mass, in a subject. Optionally, the treatment provided by the present disclosure encompasses (a) treatment or prevention of an SRE in a subject with bone metastases from solid tumors, (b) treatment or prevention of an SRE in a subject who is an adult or a skeletally mature adolescent with giant cell tumor of bone that is unresectable or where surgical resection is likely to result in severe morbidity, (c) treatment of hypercalcemia of malignancy refractory to bisphosphonate therapy in a subject, (d) treatment or prevention of an SRE in a subject with multiple myeloma or with bone metastases from a solid tumor, (c) treatment of osteoporosis of postmenopausal women at high risk for fracture, (f) treatment to increase bone mass in women at high risk for fracture receiving adjuvant aromatase inhibitor therapy for breast cancer, (g) treatment to increase bone mass in men at high risk for fracture receiving androgen deprivation therapy for nonmetastatic prostate cancer, (h) treatment to increase bone mass in men with osteoporosis at high risk for fracture, (i) therapy with calcium or vitamin D. In various instances, the disease is one described in International Patent Publication No. WO2018/200918, the entire contents of which are incorporated herein by reference.

### Subjects

In exemplar embodiments of the present disclosure, the subject is a mammal, including, but not limited to, mammals of the order Rodentia, such as mice and hamsters, and mammals of the order Logomorpha, such as rabbits, mammals from the order Carnivora, including Felines (cats) and Canines (dogs), mammals from the order Artiodactyla, including Bovines (cows) and Swines (pigs) or of the order Perssodactyla, including Equines (horses). In some aspects, the mammals are of the order Primates. Ceboids, or Simoids (monkeys) or of the order Anthropoids (humans and apes). In some aspects, the mammal is a human. In various aspects, the subject has a neoplastic disease, e.g., any one of those described herein. The term “patient”, “subject”, or “mammal” as used herein refers to any “patient”, “subject”, or “mammal” including humans, cows, horses, dogs and cats. In one embodiment of the invention, the mammal is a human. In various aspects, the subject is an adult human. In various instances, the subject has a disease described herein.

### EXEMPLARY EMBODIMENTS

Exemplary embodiments of the present invention include but are not limited to the following:
1. A liquid composition comprising (a) a monoclonal antibody at a concentration greater than about 100 mg/mL, (b) about 200 mM to about 400 mM arginine glutamate, and (c) a surfactant, wherein the pH of the liquid composition is about 4.5 to about 5.5.
2. A liquid composition comprising (a) a monoclonal antibody at a concentration greater than about 100 mg/mL, (b) about 100 mM to about 350 mM proline, (c) a buffer, and (d) a surfactant, wherein the pH of the liquid composition is about 4.5 to about 5.5.
3. The liquid composition of embodiment 1 or 2, wherein the concentration of the monoclonal antibody is less than about 300 mg/mL.
4. The liquid composition of embodiment 3, wherein the concentration of the monoclonal antibody is less than about 250 mg/mL.
5. The liquid composition of any one of embodiments 1-4, comprising about 110 mg/mL to about 200 mg/mL monoclonal antibody.
6. The liquid composition of embodiment 5, wherein the concentration of the monoclonal antibody is about 120 mg/mL to about 180 mg/mL.
7. The liquid composition of embodiment 6, comprising about 120 mg/mL monoclonal antibody.
8. The liquid composition of embodiment 6, comprising about 135 mg/mL to about 165 mg/mL monoclonal antibody.
9. The liquid composition of embodiment 8, comprising about 140 mg/mL to about 160 mg/mL monoclonal antibody.
10. The liquid composition of embodiment 9, comprising about 140 mg/mL monoclonal antibody.
11. The liquid composition of embodiment 9, comprising about 150 mg/mL monoclonal antibody.
12. The liquid composition of any one of the preceding embodiments, wherein the monoclonal antibody is an IgG1 antibody.
13. The liquid composition of any one of the preceding embodiments, wherein the monoclonal antibody binds to IL-15.
14. The liquid composition of embodiment 13, comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6.
15. The liquid composition of embodiment 13 or 14, comprising a HC variable region of SEQ ID NO: 7 and a LC variable region of SEQ ID NO: 8.
16. The liquid composition of any one of embodiments 13 to 15, comprising a HC of SEQ ID NO: 9 and a LC of SEQ ID NO: 10.
17. The liquid composition of any one of embodiments 1 to 12, wherein the monoclonal antibody binds to PD-1.
18. The liquid composition of embodiment 17, comprising a HC CDR1 of SEQ ID NO: 11, a HC CDR2 of SEQ ID NO: 12, a HC CDR3 of SEQ ID NO: 13, a LC CDR1 of SEQ ID NO: 14, a LC CDR2 of SEQ ID NO: 15, and a LC CDR3 of SEQ ID NO: 16.
19. The liquid composition of embodiment 17 or 18, comprising a HC variable region of SEQ ID NO: 17 and a LC variable region of SEQ ID NO: 18.
20. The liquid composition of any one of embodiments 17 to 19, comprising a HC of SEQ ID NO: 19 and a LC of SEQ ID NO: 20.
21. The liquid composition of embodiment 20, wherein the monoclonal antibody binds to Glucocorticoid-Induced TNFR-Related (GITR).
22. The liquid composition of embodiment 21, comprising a HC CDR1 of SEQ ID NO: 21, a HC CDR2 of SEQ ID NO: 22, a HC CDR3 of SEQ ID NO: 23, a LC CDR1 of SEQ ID NO: 24, a LC CDR2 of SEQ ID NO: 25, and a LC CDR3 of SEQ ID NO: 26.
23. The liquid composition of embodiment 21 or 22, comprising a HC variable region of SEQ ID NO: 27 and a LC variable region of SEQ ID NO: 28.
24. The liquid composition of am one of embodiments 21 to 23, comprising a HC of SEQ ID NO: 29 and a LC of SEQ ID NO: 30.
25. The liquid composition of am one of embodiments 1-11, wherein the IgG antibody is an IgG₂ antibody.
26. The liquid composition of embodiment 25, wherein the monoclonal antibody binds to RANK-L.
27. The liquid composition of embodiment 26, comprising a HC CDR1 of SEQ ID NO: 31, a HC CDR2 of SEQ ID NO: 32, a HC CDR3 of SEQ ID NO: 33, a LC CDR1 of SEQ ID NO: 34, a LC CDR2 of SEQ ID NO: 35, and a LC CDR3 of SEQ ID NO: 36.
28. The liquid composition of embodiment 26 or 27, comprising a HC variable region of SEQ ID NO: 37 and a LC variable region of SEQ ID NO: 38.
29. The liquid composition of any one of embodiments 26 to 28, comprising a HC of SEQ ID NO: 39 and a LC of SEQ ID NO: 40.
30. The liquid composition of any one of embodiments 1 and 3-29, comprising about 225 mM to about 350 mM arginine glutamate.
31. The liquid composition of any one of embodiments 1 and 3-30, comprising about 250 mM to about 325 mM arginine glutamate
32. The liquid composition of any one of embodiments 1 and 3-31, comprising about 200 mM to about 300 mM arginine glutamate
33. The liquid composition of any one of embodiments 1 and 3-31, comprising about 200 mM arginine glutamate.
34. The liquid composition of any one of embodiments 1 and 3-32, comprising a molar ratio of arginine to glutamate of about 0.7:1.0 to about 1.1:1.0, optionally, about 0.8:1.0 to about 1.1:1.0.
35. The liquid composition of any one of embodiments 1 and 3-33, wherein arginine and glutamate are the only amino acids present in the liquid composition.
36. The liquid composition of any one of the preceding embodiments, wherein the surfactant is amphipathic and/or nonionic.
37. The liquid composition of embodiment 35, wherein the surfactant is a polysorbate.
38. The liquid composition of embodiment 36, wherein the surfactant is polysorbate 20 (PS20) or polysorbate 80 (PS80) or a mixture thereof.
39. The liquid composition of any one of the preceding embodiments, comprising a surfactant at a concentration of about 0.001% (w/v) to about 0.050% (w/v).
40. The liquid composition of embodiment 39, comprising a surfactant at a concentration of about 0.005% (w/v) to about 0.025% (w/v).
41. The liquid composition of embodiment 38, comprising about 0.01% (w/v)±0.001% (w/v) surfactant.
42. The liquid composition of embodiment 39, comprising about 0.01% (w/v) polysorbate 80 (PS80).
43. The liquid composition of any one of the preceding embodiments, wherein the surfactant does not significantly alter the viscosity of the composition.
44. The liquid composition of any one of the preceding embodiments, having a pH of about 4.70 to about 5.30.
45. The liquid composition of embodiment 42, having a pH of about 5.0.
46. The liquid composition of any one of embodiments 245, comprising about 200 mM to about 30) mM proline.
47. The liquid composition of any one of embodiments 2-46, comprising about 225 mM to about 275 mM proline.
48. The liquid composition of any one of embodiments 2-47, comprising about 235 mM to about 265 mM proline.
49. The liquid composition of any one of embodiments 2-48, comprising about 240 mM to about 260 mM proline.
50. The liquid composition of any one of embodiments 2-49, wherein the proline is L-proline.
51. The liquid composition of any one of embodiments 2-50, wherein proline is the only amino acid present in the composition.
52. The liquid composition of any one of embodiments 2-51, wherein the buffer is selected from the group consisting of: succinate, glutamate, histidine, and acetate.
53. The liquid composition of embodiment 52, wherein the buffer is acetate.
54. The liquid composition of embodiment 53, wherein the acetate is prepared with glacial acetic acid.
55. The liquid composition of any one of embodiments 2-54, wherein the buffer is prepared with about 1 mM to about 50 mM buffer, optionally, glacial acetic acid.
56. The liquid composition of embodiment 55, wherein the buffer is prepared with about 18 mM to about 22 mM buffer, optionally, 20 mM glacial acetic acid.
57. The liquid composition of any one of embodiments 2-51, comprising about 30 mM to about 38 mM buffer, optionally, about 34 mM acetate.
58. The liquid composition of any one of 52-57, wherein the pH of the buffer is titrated with sodium hydroxide.
59. The liquid composition of any one of the preceding embodiments, comprising not more than 0.001% (w/v) of a sugar, sugar alcohol, or citrate.
60. The liquid composition of any one of the preceding embodiments, comprising not more than 0.001% (w/v) disaccharide.
61. The liquid composition of any one of the preceding embodiments, comprising not more than 0.001% (w/v) trehalose or sucrose.
62. The liquid composition of any one of embodiments 1, 345, and 59-61 comprising less than about 0.001% (w/v) acetate.
63. The liquid composition of any one of embodiments 1, 345 and 59-62, comprising less than about 0.001% (w/v) a buffer.
64. The liquid composition of any one of the preceding embodiments, wherein the viscosity of the liquid composition is less than 50 cP at 25° C., 1000 s⁻¹.
65. The liquid composition of embodiment 65, wherein the viscosity of the liquid composition is less than 30 cP at 25° C., 1000 s⁻¹.
66. The liquid composition of embodiment 66, wherein the viscosity of the liquid composition is less than 20 cP at 25° C., 1000 s⁻¹.
67. The liquid composition of embodiment 67, wherein the viscosity of the liquid composition is less than or about 10 cP at 25° C., 1000 s⁻¹.
68. The liquid composition of any one of the preceding embodiments, wherein the composition is isotonic.
69. The liquid composition of am one of the preceding embodiments, wherein the composition has an osmolality in a range of about 200 mOsm/kg to about 500 mOsm/kg, optionally, about 225 mOsm/kg to about 400 mOsm/kg, or.
70. The liquid composition of embodiment 69, wherein the composition has an osmolality in a range of about about 250 mOsm/kg to about 350 mOsm/kg.
71. The liquid composition of any one of the preceding embodiments, wherein less than 5% of the antibody is degraded after storage at 2° C. to 8° C. for at least or about 12 months or about 20 months to about 26 months, as determined by Size Exclusion Chromatography (SEC).
72. The liquid composition of any one of the preceding embodiments, wherein less than 5% of the antibody is degraded after storage at 2° C. to 8° C. for about 30 to about 40 months, as determined by SEC.
73. The liquid composition of any one of the preceding embodiments, wherein less than 5% of the antibody is degraded after storage at 2° C. to 8° C. for about 36 40 months, as determined by SEC.
74. A method of preparing a liquid composition comprising a target concentration of a monoclonal antibody, wherein the target concentration is greater than about 100 mg/mL, said method comprising (a) combining the monoclonal antibody with a diafiltration (DF) buffer comprising (i) about 50 mM to about 300 mM arginine base and (ii) an amount of glutamate to achieve a molar ratio of arginine to glutamate of about 0.7:1.0 to about 1.1:1.0, and (b) adding a surfactant.
75. The method of embodiment 74, wherein the molar ratio is about 0.8:1.0 to about 1.1:1.0.
76. The method of embodiment 74 or 75, wherein the DF buffer comprises about 85 mM to about 190 mM L-arginine.
77. The method of any one of embodiments 74 to 76, wherein the DF buffer comprises about 135 mM to about 165 mM arginine base.
78. The method of any one of embodiments 74 to 77, wherein the DF buffer comprises about 155 mM to about 185 mM glutamate.
79. The method of embodiment 78, wherein the DF buffer comprise about 150 mM arginine base and about 170 mM glutamate.
80. The method of any one of embodiments 74-79, wherein the pH of the DF buffer is about the same as the final pH of the prepared liquid composition.
81. The method of any one of embodiments 74 to 80, wherein the final pH of the prepared liquid composition is about 4.5 to about 5.5, optionally, about 4.7 to about 5.3.
82. The method of any one of embodiments 74-81, wherein the surfactant is polysorbate 80.
83. The method of any one of embodiments 74 to 82, wherein the final concentration of the surfactant is about 0.01% (w/v).
84. A liquid composition prepared by the method of any one of embodiments 74-83.
85. A liquid composition comprising, per mL liquid composition, (a) about 150 mg to about 165 mg monoclonal antibody, (b) about 22 mg to about 26 mg L-arginine, (c) about 22 mg to about 26 mg glutamic acid, and (d) about 0.01 mg PS80, wherein the liquid composition has a pH of about 4.7 to about 5.3.
86. The liquid composition of embodiment 84, comprising about 24 mg L-arginine.
87. The liquid composition of embodiment 84 or 85, comprising about 23 mg glutamic acid.
88. A liquid composition comprising, per mL liquid composition, about 150 mg to about 165 mg monoclonal antibody in about 180 mM to about 220 mM proline, about 30 mM to about 38 mM acetate, and about 0.01% (w/v) PS80, wherein the liquid composition has a pH of about 4.7 to about 5.3.
89. The liquid composition of embodiment 87, comprising about 34 mM acetate.
90. The liquid composition of embodiment 87 or 88, comprising about 200 mi proline.
91. The liquid composition of any one of embodiments 84-89, wherein the pH is about 5.0.
92. A liquid composition comprising (a) an anti-IL-15 antibody at a concentration greater than about 10) mg/mL, (b) about 70 mM to about 210 mM arginine base, (c) about 80 mM to about 240 mM glutamate, and (d) a surfactant, wherein the pH of the liquid composition is about 4.5 to about 5.5.
93. The liquid composition of embodiment 92, comprising about 100 mM to about 170 mM arginine base.
94. The liquid composition of embodiment 93, comprising about 120 mM to about 150 mM arginine base.
95. The liquid composition of embodiment 94, comprising about 136 mM arginine base.
96. The liquid composition of any one of embodiments 92-95, comprising about 120 mM to about 200 mM glutamate.
97. The liquid composition of embodiment 96, comprising about 140 mM to about 175 mM glutamate.
98. The liquid composition of embodiment 97, comprising about 159 mM glutamate.
99. The liquid composition of any one of embodiments 92-98, wherein arginine and glutamate are the only amino acids present in the liquid composition.
100. The liquid composition of any one of embodiments 92-99 comprising less than about 0.001% (w/v) acetate.
101. The liquid composition of any one of embodiments 92-100, comprising less than about 0.001% (w/v) a buffer.
102. A liquid composition comprising (a) an anti-IL-15 antibody at a concentration greater than about 100 mg/mL, (b) about 115 mM to about 345 mM proline, (c) a buffer, (d) a surfactant, wherein the pH of the liquid composition is about 4.5 to about 5.5.
103. The liquid composition of embodiment 102, comprising about 170 mM to about 290 mM proline.
104. The liquid composition of embodiment 103, comprising about 200 mM to about 255 mM proline.
105. The liquid composition of embodiment 104, comprising about 230 mM proline.
106. The liquid composition of any one of embodiments 102-105, wherein proline is the only amino acid present in the composition.
107. The liquid composition of any one of embodiments 102-106, comprising about 1 mM to about 100 mM buffer, optionally, about 1 mM to about 50 mM buffer.
108. The liquid composition of embodiment 107, comprising about 10 mM to about 30 mM buffer, optionally, about 15 mM to about 25 mM buffer.
109. The liquid composition of any one of embodiments 102-108, wherein the buffer is selected from the group consisting of: succinate, glutamate, histidine, and acetate.
110. The liquid composition of embodiment 109, wherein the buffer is acetate.
111. The liquid composition of any one of embodiments 92-110, comprising not more than 0.001% (w/v) of a sugar, sugar alcohol, or citrate.
112. The liquid composition of any one of embodiments 92-111, comprising not more than 0.001% (w/v) disaccharide.
113. The liquid composition of any one of embodiments 92-112, comprising not more than 0.001% (w/v) trehalose or sucrose.
114. The liquid composition of any one of embodiments 92-113, wherein the concentration of the anti-IL-15 antibody is less than about 300 mg/mL.
115. The liquid composition of embodiment 114, wherein the concentration of the anti-IL-15 antibody is less than about 250 mg/mL.
116. The liquid composition of any one of embodiments 92-115, comprising about 110 mg/mL to about 200 mg/mL anti-IL-15 antibody.
117. The liquid composition of embodiment 116, comprising about 135 mg/mL to about 165 mg/mL anti-IL-15 antibody.
118. The liquid composition of embodiment 117, comprising about 140 mg/mL to about 160 mg/mL anti-IL-15 antibody.
119. The liquid composition of embodiment 118, comprising about 150 mg/mL anti-IL-15 antibody.
120. The liquid composition of any one of embodiments 92-116, comprising about 145 mg/mL to about 182 mg/mL anti-IL-15 antibody.
121. The liquid composition of embodiment 120, comprising about 155 mg/mL to about 175 mg/mL anti-IL-15 antibody.
122. The liquid composition of embodiment 121, comprising about 165 mg/mL anti-IL-15 antibody.
123. The liquid composition of any one of embodiments 92-122, wherein the anti-IL-15 antibody is an IgG₁ antibody.
124. The liquid composition of embodiment 123, comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6.
125. The liquid composition of embodiment 123 or 124, comprising a HC variable region of SEQ ID NO: 7 and a LC variable region of SEQ ID NO: 8.
126. The liquid composition of any one of embodiments 123 to 125, comprising a HC of SEQ ID NO: 9 and a LC of SEQ ID NO: 10.
127. The liquid composition of any one of embodiments 92-126, wherein the surfactant is amphipathic and/or nonionic.
128. The liquid composition of embodiment 127, wherein the surfactant is a polysorbate.
129. The liquid composition of embodiment 128, wherein the surfactant is polysorbate 20 (PS20) or polysorbate 80 (PS80) or a mixture thereof.
130. The liquid composition of am one of embodiments 92-1129, comprising a surfactant at a concentration of about 0.001% (w/v) to about 0.050% (w/v).
131. The liquid composition of embodiment 130, comprising a surfactant at a concentration of about 0.005% (w/v) to about 0.025% (w/v).
132. The liquid composition of embodiment 131, comprising about 0.01% (w/v)±0.001% (w/v) surfactant.
133. The liquid composition of embodiment 132, comprising about 0.01% (w/v) polysorbate 80 (PS80).
134. The liquid composition of any one of embodiments 92-133, wherein the surfactant does not significantly alter the viscosity of the composition.
135. The liquid composition of any one of embodiments 92-134, having a pH of about 4.6 to about 5.4.
136. The liquid composition of embodiment 135, having a pH of about 4.70 to about 5.30.
137. The liquid composition of embodiment 136, having a pH of about 4.8 to about 5.2
138. The liquid composition of embodiment 137, having a pH of about 4.9 to about 5.1.
139. The liquid composition of embodiment 138, having a pH of about 5.0.
140. The liquid composition of any one of embodiments 92-139, wherein the viscosity of the liquid composition is less than 50 cP at 25° C., 1000 s⁻¹.
141. The liquid composition of embodiment 140, wherein the viscosity of the liquid composition is less than 30 cP at 25° C., 1000 s⁻¹.
142. The liquid composition of embodiment 141, wherein the viscosity of the liquid composition is less than 20 cP at 25° C., 1000 s⁻¹.
143. The liquid composition of embodiment 142, wherein the viscosity of the liquid composition is less than or about 10 cP at 25° C., 1000 s⁻¹.
144. The liquid composition of any one of embodiments 92-143, wherein the composition is isotonic.
145. The liquid composition of any one of embodiments 92-143, wherein the composition has an osmolality in a range of about 200 mOsm/kg to about 500 mOsm/kg, optionally, about 225 mOsm/kg to about 400 mOsm/kg, or.
146. The liquid composition of embodiment 145, wherein the composition has an osmolality in a range of about about 250 mOsm/kg to about 350 mOsm/kg.
147. The liquid composition of any one of embodiments 92-146, wherein less than 5% of the antibody is degraded after storage at 2° C. to 8° C. for at least or about 12 months or about 20 months to about 26 months, as determined by Size Exclusion Chromatography (SEC).
148. The liquid composition of am one of embodiments 92-147, wherein less than 5% of the antibody is degraded after storage at 2° C. to 8° C. for about 30 to about 40 months, as determined by SEC.
149. The liquid composition of am one of embodiments 92-148, wherein less than 5% of the antibody is degraded after storage at 2° C. to 8° C. for about 36 40 months, as determined by SEC.
150. A liquid composition comprising, per mL of the liquid composition, (i) about 135 mg to about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, (ii) about 21 mg to about 26 mg arginine base, (iii) about 21 mg to about 26 mg glutamate, and (iv) about 0.01% (w/v) polysorbate 80 (PS80), wherein the liquid composition has a pH of about 4.5 to about 5.5.
151. A liquid composition comprising, per mL of the liquid composition, (i) about 135 mg to about 165 ng of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6, (ii) about 23 mg to about 30 mg proline, (iii) about 0.5 mg to about 2 mg acetate, and (iv) about 0.01% (w/v) polysorbate 80 (PS80), wherein the liquid composition has a pH of about 4.5 to about 5.5.
152. A method of preparing a liquid composition comprising a target concentration of an anti-IL-15 antibody, wherein the target concentration is greater than about 100 mg/mL, said method comprising (a) combining the antibody with a diafiltration (DF) buffer comprising about 70 mM to about 210 mM arginine base and about 80 mM to about 240 mM glutamate, and (b) adding a surfactant.
153. The method of embodiment 152, wherein the DF buffer comprises about 100 mM to about 170 mM arginine base.
154. The method of embodiment 153, wherein the DF buffer comprises about 120 mM to about 150 mM arginine base.
155. The method of embodiment 154, wherein the DF buffer comprises about 136 mM arginine base.
156. The method of any one of embodiments 152-154, wherein the DF buffer comprises about 120 mM to about 200 mM glutamate.
157. The method of embodiment 156, wherein the DF buffer comprises about 140 mM to about 175 mM glutamate.
158. The method of embodiment 157, wherein the DF buffer comprises about 159 mM glutamate.
159. The method of any one of embodiments 152-158, wherein arginine and glutamate are the only amino acids present in the DF buffer.
160. The method of any one of embodiments 152-159, wherein the pH of the DF buffer is about the same as the final pH of the prepared liquid composition.
161. The method of any one of embodiments 152 to 160, wherein the final pH of the prepared liquid composition is about 4.5 to about 5.5, optionally, about 4.7 to about 5.3 or about 4.8 to about 5.2.
162. The method of any one of embodiments 152-161, wherein the surfactant is polysorbate 80.
163. The method of any one of embodiments 152 to 162, wherein the final concentration of the surfactant is about 0.01% (w/v).
164. A liquid composition prepared by the method of any one of embodiments 152-163.
165. An article of manufacture comprising the liquid composition of any one of the preceding embodiments, optionally, comprising about 1 mL to about 5 mL of the liquid composition.
166. A pre-filled syringe comprising the liquid composition of any one of the preceding embodiments, optionally, comprising about 1 mL to about 5 mL of the liquid composition.
167. A vial comprising the liquid composition of any one of the preceding embodiments, optionally, comprising about 1 mL to about 5 mL of the liquid composition.
168. An autoinjector comprising the liquid composition of any one of the preceding embodiments.
169. A method of treating a disease in a subject, comprising administering to the subject a liquid composition of any one of the preceding embodiments in an amount effective to treat the disease.
170. Use of the liquid composition of any one of the preceding embodiments for treating a disease.
171. The method of embodiment 169 or the use of embodiment 170, wherein the disease is a celiac disease.

The following examples are given merely to illustrate the present invention and not in any way to limit its scope.

### EXAMPLES

### Example 1

This example demonstrates an exemplary method of producing a high concentration antibody formulation.

A series of studies were carried out to develop an isotonic, low viscosity formulation containing a high concentration of Antibody 1, an anti-IL-15 monoclonal antibody. The formulation was designed to demonstrate suitable stability, as characterized by low initial high molecular weight (HMW) species formation, as well low HMW species formation upon storage.

### Part A

In the first study (Part A), eight formulations were prepared by diafiltering an initial solution comprising Antibody 1, acetate and sucrose against a diafiltration (DF) buffer. A total of 10 buffer changes were carried out to achieve a complete buffer exchange. Using an ultrafiltration/diafiltration (UF/DF) system, the buffer-exchanged Antibody 1 solution was concentrated to about 200 mg/mL Antibody 1. The Antibody 1 solution was then diluted to achieve the target concentration of 165 mg/mL using the same DF buffer used in the buffer exchange step. Polysorbate 80 (PS80) was then added to a final concentration of 0.01% (w/v). Table 1 summarizes the DF buffer used to make each formulation and the final pH of each formulation. The osmolality for each formulation was measured and found to be within a range of about 284 mOsm/kg to about 332 mOsm/kg.

TABLE 1

| | | | Final |
|---|---|---|---|
| Formulation | | DF Buffer | Formulation |
| No. | DF Buffer Components | pH | pH |
| 1 | 20 mM acetate | 4.8 | 5.2 |
| | 7% (w/v) sucrose | | |
| 2 | 20 mM acetate | 5.0 | 5.2 |
| | 150 mM NAR | | |
| | 100 mM L-proline | | |
| 3 | 20 mM acetate | 4.9 | 5.2 |
| | 250 mM L-proline | | |
| 4 | 20 mM acetate | 5.1 | 5.2 |
| | 150 mM L-arginine HCl | | |
| 5 | 20 mM acetate | 5.2 | 5.2 |
| | 150 mM L-arginine base | | |
| | 150 mM L-glutamic acid | | |
| 6 | 20 mM acetate | 4.9 | 4.7 |
| | 40 mM L-phenylalanine | | |
| | 7% (w/v) sucrose | | |
| 7 | 20 mM acetate | 3.5 | 4.7 |
| | 7% (w/v) sucrose | | |
| 8 | 20 mM acetate | 5.2 | 5.5 |
| | 7% (w/v) sucrose | | |

NAR, N-acetyl arginine

### Stability Testing:

A sample of each formulation was filled into a container, sealed and then stored for 0, 4, 13, or 26 weeks at 2-8° C. or for 1, 2, 4, 13 or 26 weeks at 30° C. Initial formation of HMW species was measured and reported as t=0. Samples were tested via size exclusion-ultra high-performance liquid chromatography (SE-UHPLC). Percentage of the high molecular weight (HMW) species for samples was reported and reflected the amount of HMW species that formed after the storage period. The results of the stability assay are shown in Tables 2 and 3. A percent HMW species is shown for each sample. The lower the % HMW indicated greater stability for the formulation.

TABLE 2
Storage Temperature = 2-8° C.

| | Storage Time, t | |
|---|---|---|
| Formulation No. | (weeks) | % HMW |
| 1 | 0 | 1.9 |
| | 4 | 2.4 |
| | 13 | 2.7 |
| | 26 | 2.9 |
| 2 | 0 | 2.0 |
| | 4 | 2.3 |
| | 13 | 2.6 |
| | 26 | 2.9 |
| 3 | 0 | 1.9 |
| | 4 | 2.3 |
| | 13 | 2.6 |
| | 26 | 2.8 |
| 4 | 0 | 1.6 |
| | 4 | 1.9 |
| | 13 | 2.3 |
| | 26 | 2.6 |
| 5 | 0 | 1.3 |
| | 4 | 1.6 |
| | 13 | 1.9 |
| | 26 | 2.1 |
| 6 | 0 | 1.7 |
| | 4 | 2.0 |
| | 13 | 2.3 |
| | 26 | 2.6 |
| 7 | 0 | 1.5 |
| | 4 | 1.8 |
| | 13 | 2.1 |
| | 26 | 2.4 |
| 8 | 0 | 2.4 |
| | 4 | 3.0 |
| | 13 | 3.5 |
| | 26 | 3.9 |

TABLE 3
Storage Temperature = 30° C.

| | Storage Time, t | |
|---|---|---|
| Formulation No. | (weeks) | % HMW |
| 1 | 0 | 1.9 |
| | 1 | 2.6 |
| | 2 | 3.2 |
| | 4 | 3.6 |
| | 13 | 5.0 |
| | 26 | 6.3 |
| 2 | 0 | 2.0 |
| | 1 | 2.6 |
| | 2 | 3.0 |
| | 4 | 3.3 |
| | 13 | 4.1 |
| | 26 | 4.8 |
| 3 | 0 | 1.9 |
| | 1 | 2.6 |
| | 2 | 3.0 |
| | 4 | 3.4 |
| | 13 | 4.5 |
| | 26 | 5.6 |
| 4 | 0 | 1.6 |
| | 1 | 2.1 |
| | 2 | 2.5 |
| | 4 | 2.9 |
| | 13 | 4.1 |
| | 26 | 5.1 |
| 5 | 0 | 1.3 |
| | 1 | 1.8 |
| | 2 | 2.1 |
| | 4 | 2.5 |
| | 13 | 3.7 |
| | 26 | 4.8 |
| 6 | 0 | 1.7 |
| | 1 | 2.2 |
| | 2 | 2.7 |
| | 4 | 3.2 |
| | 13 | 4.5 |
| | 26 | 5.7 |
| 7 | 0 | 1.5 |
| | 1 | 2.1 |
| | 2 | 2.4 |
| | 4 | 2.8 |
| | 13 | 4.1 |
| | 26 | 5.6 |
| 8 | 0 | 2.4 |
| | 1 | 3.3 |
| | 2 | 3.8 |
| | 4 | 4.6 |
| | 13 | 6.2 |
| | 26 | 7.4 |

### Viscosity:

Samples of each formulation were tested for viscosity using a using a viscometer at 5° C., 15° C., 20° C., 25° C. and 30° C. Reported viscosity values are at a shear rate of 1000 s⁻¹. Table 4 provides the results of this assay.

TABLE 4

| | Viscosity (cP) | | | | |
|---|---|---|---|---|---|
| Formulation No. | 5° C. | 15° C. | 20° C. | 25° C. | 30° C. |
| 1 | 25.0 | 16.3 | 15.4 | 12.6 | 9.5 |
| 2 | 17.3 | 13.3 | 11.7 | 9.5 | 8.1 |
| 3 | 18.9 | 14.3 | 11.6 | 10.3 | 8.8 |
| 4 | 24.4 | 15.3 | 13.5 | 10.9 | 8.8 |
| 5 | 16.8 | 12.8 | 10.7 | 10.0 | 9.4 |
| 6 | 23.4 | 14.7 | 13.8 | 12.2 | 7.8 |
| 7 | 20.2 | 16.2 | 13.8 | 10.9 | 9.2 |
| 8 | 30.1 | 23.6 | 18.6 | 15.9 | 13.9 |

### Freeze-Thaw Stability

To test the stability of formulations after a freeze-thaw cycle, formulations were filled into vials and frozen at −30° C. Vials then went through 5 freeze/thaw (F/T) cycles, wherein freezing occurred at (−30) ° C. and thaw occurring statically at room temperature. Each container was visually inspected for color and presence of precipitates. After 5 FIT cycles, most formulations appeared clear and free from visible particles, however Formulation No. 2 exhibited numerous small, translucent, crystalline particles and Formulation No. 4 exhibited a small number of small fibrous particles.

### Discussion of the Results: Part A

Formulation No. 5 comprising arginine glutamate demonstrated very high stability, as this formulation had the lowest amount of initial HMW species at both 30° C. and 2-8° C., as well as the lowest amount of HMW species formed after 26 weeks of storage at each of the temperatures (Tables 2 and 3). As shown in Table 4, Formulation 5 comprising arginine glutamate demonstrated among the lowest viscosities across a range of temperatures (5° C.-25° C.).

Like Formulation No. 5, Formulation No. 2 comprising NAR and proline demonstrated very high stability after storage for 26 weeks at 30° C. However, as discussed above, this formulation was observed to have a high amount of precipitates and cloudiness upon freeze-thaw cycles. Thus, this formulation was among those not selected for subsequent studies.

Formulation 3 comprising proline (without NAR) also exhibited low % HMW after 26 weeks of storage. Formulation 3 additionally was one of the formulations that demonstrated reduced viscosities across a range of temperatures.

### Part B

In the second study (Part B), seven formulations of Antibody 1 were prepared as essentially described above. This study tested two target concentrations of Antibody 1 (150 mg/mL and 165 mg/mL) and two pHs (4.7 and 5.2). PS80 was added to a final concentration of 0.01% for all formulations except for Formulation 5b, which had a final PS80 concentration of 0.005% (w/v). Table 5 summarizes the DF buffer used to make each formulation. The osmolality for each formulation was measured and found to be within a range of about 280 mOsm/kg to about 331 mOsm/kg.

TABLE 5

| Formula- | | DF | PS80 | Final Ab1 | |
|---|---|---|---|---|---|
| tion | | Buffer | (% | Concentra- | |
| No. | DF Buffer Components | pH | w/v) | tion | pH |
| 1b | 20 mM acetate | 4.9 | 0.01 | 150 | 5.2 |
| | 7% (w/v) sucrose | | | | |
| 2b | 20 mM acetate | 3.6 | 0.01 | 150 | 4.7 |
| | 7% (w/v) sucrose | | | | |
| 3b | 150 mM L-arginine base, | 5.2 | 0.01 | 150 | 5.2 |
| | 166 mM L-glutamic acid | | | | |
| 4b | 128 mM L-arginine base, | 4.7 | 0.01 | 150 | 4.7 |
| | 169 mM L-glutamic acid | | | | |
| 5b | 128 mM L-arginine base, | 4.7 | 0.005 | 165 | 4.7 |
| | 169 mM L-glutamic acid | | | | |
| 6b | 20 mM acetate | 4.0 | 0.01 | 150 | 4.7 |
| | 250 mM proline | | | | |
| 7b | 20 mM acetate | 4.0 | 0.01 | 150 | 5.2 |
| | 250 mM proline | | | | |

The stability of each formulation was tested as essentially described in Part A, while the viscosity was tested at shear rate of 50800 s⁻¹ to mimic shear rates associated with manual extrusion of a PFS. The stability upon storage at 40° C. was also tested in this study (Part B). The viscosity at 5° C. and 25° C. for two Antibody 1 concentrations (150 mg/mL and 165 mg/mL) were additionally tested. The results of the stability assay are reported in Tables 6, 7, and 8, an viscosity results are shown in Table 9.

TABLE 6
Storage Temperature = 2-8° C.

| Formulation No | 0 weeks | 4 weeks | 13 weeks | 26 weeks |
|---|---|---|---|---|
| 1b | 1.8 | 2.0 | 2.3 | 2.6 |
| 2b | 1.4 | 1.5 | 1.8 | 2.0 |
| 3b | 1.2 | 1.3 | 1.6 | 1.8 |
| 4b | 1.0 | 1.2 | 1.3 | 1.5 |
| 5b | 1.1 | 1.2 | 1.4 | 1.6 |
| 6b | 1.3 | 1.4 | 1.7 | 1.8 |
| 7b | 1.6 | 1.9 | 2.1 | 2.3 |

TABLE 7
Storage Temperature = 30° C.

| Formulation | 0 | 1 | 2 | 4 | 13 | 26 |
|---|---|---|---|---|---|---|
| No | weeks | week | weeks | weeks | weeks | weeks |
| 1b | 1.8 | 2.6 | 3.0 | 3.5 | 4.8 | 6.2 |
| 2b | 1.4 | 1.9 | 2.2 | 2.6 | 3.9 | 5.4 |
| 3b | 1.2 | 1.7 | 1.9 | 2.2 | 3.2 | 4.3 |
| 4b | 1.0 | 1.4 | 1.6 | 1.9 | 2.9 | 4.1 |
| 5b | 1.1 | 1.5 | 1.7 | 2.0 | 3.1 | 4.5 |
| 6b | 1.3 | 1.8 | 2.0 | 2.4 | 3.3 | 4.4 |
| 7b | 1.6 | 2.3 | 2.6 | 3.0 | 4.1 | 5.1 |

TABLE 8
Storage Temperature = 40° C.

| Formulation | 0 | 0.4 | 1 | 2 | 4 | 13 |
|---|---|---|---|---|---|---|
| No | weeks | week | week | weeks | weeks | weeks |
| 1b | 1.8 | 2.7 | 3.1 | 3.8 | 4.7 | 8.0 |
| 2b | 1.4 | 2.0 | 2.4 | 3.0 | 3.9 | 8.1 |
| 3b | 1.2 | 1.7 | 2.0 | 2.5 | 3.3 | 6.2 |
| 4b | 1.0 | 1.4 | 1.8 | 2.3 | 3.2 | 7.2 |
| 5b | 1.1 | 1.5 | 1.9 | 2.5 | 3.3 | 7.2 |
| 6b | 1.3 | 1.9 | 2.2 | 2.7 | 3.4 | 6.3 |
| 7b | 1.6 | 2.4 | 2.8 | 3.4 | 4.2 | 6.8 |

TABLE 9

| | [Antibody 1] = 150 mg/mL | | [Antibody 1] = 165 mg/mL | |
|---|---|---|---|---|
| | 5° C. | 25° C. | 5° C. | 25° C. |
| 1b | 16.3 | 7.82 | 24.9 | 10.8 |
| 2b | 14.5 | 7.4 | 21.9 | 9.2 |
| 3b | 14.1 | 6.9 | 20.6 | 9.1 |
| 4b | 12.0 | 6.0 | 18.2 | 8.2 |
| 5b | 12.8 | 6.5 | 17.6 | 7.9 |
| 6b | 11.7 | 5.9 | 16.7 | 7.7 |
| 7b | 13.0 | 6.5 | 18.5 | 8.5 |

### Discussion of the Results: Part B

As shown in Tables 6-8, Formulations 2b-5b comprising arginine glutamate as well as Formulation 6b comprising proline demonstrated lower % HMW species after storage for 26 weeks at 2-8° C. or 30° C. or after storage for 13 weeks at 40° C., compared to Formulations 1b and 2b comprising acetate and sucrose. Formulations 3b and 4b each comprising arginine glutamate and differing only by pH, performed particularly well among all formulations tested. As shown in Table 9, each of Formulations 2b-5b comprising arginine glutamate as well as Formulations 6b and 7b comprising proline demonstrated lower viscosities, compared to Formulations 1b and 2b. Formulation 6b demonstrated the lowest viscosities at both antibody concentrations and at both temperatures. Also, interestingly, the performance of the arginine glutamate did not appear to depend on acetate, suggesting that the glutamate may be dually functioning as both a counter ion to arginine and as a buffering agent.

### Conclusions

Taken together, these data suggest that Antibody 1 at a concentration greater than 100 mg/mL formulated with about 100 mM to about 2N00 mM L-arginine base and about 100 mM to about 200 mM L-glutamic acid, PS80 (final concentration of 0.010% (w/v)), pH 4.7 to 5.2, exhibits high stability, as evidenced by low initial HMW species formation and by low HMW species formation upon storage, and reduced viscosity. It was surprising that the amounts of arginine and glutamate used in these studies led to such low viscosities (<10 cP at 25° C.), given that similar formulations described in previous studies (e.g., Bonvankar et al., Ind. Eng. Chem Res. 55: 11225-11234 (2016)) required very high amounts of the salt (e.g., 450 mM) to achieve similar viscosities. Formulations comprising similar amounts of arginine glutamate in previous studies had viscosities of about 170 cP.

The high stability and low viscosity of Antibody 1 at a concentration greater than 100 mg/mL formulated with about 150 mM to about 250 mM proline and about 20 mM acetate also was surprising, given that previous studies have shown that Pro, only at very high concentrations which are too impractical, achieve an acceptable level of viscosity reduction (Hung et al., Pharm Res 35: 133 (2018)).

In view of the foregoing, formulations comprising arginine glutamate or proline and acetate were selected for further testing and development of a formulation comprising 150 mg/mL Antibody 1.

### Example 2

This example demonstrates that antibodies other than Antibody 1 exhibit high stability and low viscosity when formulated with arginine base and glutamic acid.

In this study, five different antibodies were formulated at different concentrations (100 mg/mL, 150 mg/mL, and 200 mg/mL) with either a Test DF buffer comprising L-arginine base and L-glutamic acid or a Control DF buffer comprising acetate and sucrose. The formulations were prepared as essentially described in Example 1. PS80 was added after UF/DF to achieve a final concentration of 0.01% (w/v). Antibody 1 is the same as that described in Example 1. Antibody 2 is an anti-PD-1 IgG1 antibody, Antibody 3 is an anti-RANKL IgG2 antibody, Antibody 4 is an anti-GITR IgG1 antibody, and Antibody 5 is a reference IgG2 antibody which binds to an antigen different from any of Antibodies 1-4. The components of the test DF buffer used for each of the five antibodies and the five control DF buffers are summarized in Table 10.

TABLE 10

| | | DF Buffer | Final |
|---|---|---|---|
| | Components | pH | pH |
| Test DF Buffer (Antibody 1-5) | ~150 mM L-arginine base, ~170 | 5.2 | 5.2 |
| | mM L-glutamic acid | | |
| Control DF Buffer (Antibody 1) | 20 mM acetate, 8% (w/v) sucrose | 4.7 | 5.2 |
| Control DF Buffer (Antibody 2) | 20 mM acetate, 8% (w/v) sucrose | 4.6 | 5.2 |
| Control DF Buffer (Antibody 3) | 20 mM acetate, 8% (w/v) sucrose | 4.6 | 5.2 |
| Control DF Buffer (Antibody 4) | 25 mM acetate, 8% (w/v) sucrose | 4.4 | 5.2 |
| Control DF Buffer (Antibody 5) | 20 mM acetate, 8% (w/v) sucrose | 4.7 | 5.2 |

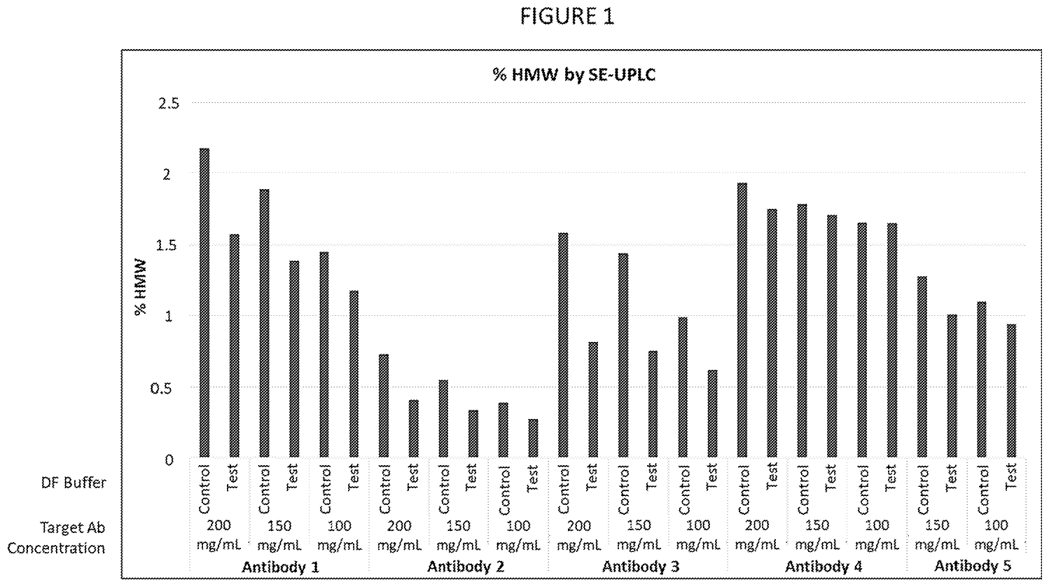

The stability after 0 weeks of storage, 2 weeks of storage at 30° C., and after 1 week of storage at 40° C. were tested via SE-UHPLC, and viscosity was measured as essentially described in Example 1. A summary of the results of the stability assay for each antibody expressed as % HMW is provided below in Table 11 and FIG. 1 and the results of the viscosity assay are provided in Table 12.

TABLE 11

| | Antibody 1 | | | Antibody 2 | | | Antibody 3 | | |
|---|---|---|---|---|---|---|---|---|---|
| | | t = 2 w | t = 1 w | | t = 2 w | t = 1 w | | t = 2 w | t = 1 w |
| Formulation | t = 0 | 30° C. | 40° C. | t = 0 | 30° C. | 40° C. | t = 0 | 30° C. | 40° C. |
| 200 mg/mL Control | 2.2 | 3.4 | 3.9 | 0.7 | 1.0 | 1.3 | 1.6 | 2.2 | 2.5 |
| 200 mg/mL Test | 1.6 | 2.4 | 2.9 | 0.4 | 0.7 | 0.9 | 0.8 | 1.3 | 1.4 |
| 150 mg/mL Control | 1.9 | 2.8 | 3.3 | 0.5 | 0.7 | 0.9 | 1.4 | 1.9 | 2.3 |
| 150 mg/mL Test | 1.4 | 2.1 | 2.5 | 0.3 | 0.5 | 0.7 | 0.8 | 1.0 | 1.3 |
| 100 mg/mL Control | 1.4 | 1.9 | 2.1 | 0.4 | 0.5 | 0.6 | 1.0 | 1.2 | 1.4 |
| 100 mg/mL Test | 1.2 | 1.5 | 1.8 | 0.3 | 0.4 | 0.5 | 0.6 | 0.7 | 0.9 |

| | Antibody 4 | | | Antibody 5 | | |
|---|---|---|---|---|---|---|
| | | t = 2 w | t = 1 w | | t = 2 w | t = 1 w |
| | t = 0 | 30° C. | 40° C. | t = 0 | 30° C. | 40° C. |
| 200 mg/mL Control | 1.9 | 2.1 | 2.1 | nd | nd | nd |
| 200 mg/mL Test | 1.8 | 1.9 | 1.9 | nd | nd | nd |
| 150 mg/mL Control | 1.8 | 1.9 | 1.8 | 1.3 | 1.6 | 3.1 |
| 150 mg/mL Test | 1.7 | 1.9 | 1.9 | 1.0 | 1.4 | 3.6 |
| 100 mg/mL Control | 1.7 | 1.6 | 1.4 | 1.1 | 1.3 | 2.1 |
| 100 mg/mL Test | 1.7 | 1.8 | 1.8 | 0.9 | 1.2 | 2.7 |

TABLE 12

| | | | Measured Protein | Viscosity cP | |
|---|---|---|---|---|---|
| | DF | Target Protein | Concentration at | (1000/sec) | |
| Antibody | Buffer | Concentration | t = 0 (mg/mL) | 5° C. | 25° C. |
| 1 | Control | 200 | 178.7 | 31.7 | 14.0 |
| 1 | Test | 200 | 192.0 | 29.3 | 14.9 |
| 2 | Control | 200 | 193.1 | 21.7 | 13.1 |
| 2 | Test | 200 | 183.2 | 15.9 | 9.3 |
| 3 | Control | 200 | 170.8 | 13.3 | 7.7 |
| 3 | Test | 200 | 171.1 | 11.0 | 6.2 |
| 4 | Control | 200 | 187.2 | 69.1 | 16.7 |
| 4 | Test | 200 | 186.9 | 80.9 | 18.9 |
| | Control | 150 | 157.8 | 166.3 | 38.6 |
| | Test | 150 | 147.7 | 23.9 | 11.6 |

As shown in Table 11, formulating the antibodies with arginine glutamate led to improved stability at an antibody concentration of 200 mg/mL, compared to the Control DF buffer, and the effect was observed across all molecules and storage temperatures. The impact of the arginine glutamate formulation lowers initial % HMW across nearly all 5 mAbs at 3 concentrations compared to the Control DF buffer.

For three of the antibodies (Antibodies 1-3), the trends were fairly consistent with regard to reduced % HMW in Arg-Glu compared to Control DF Buffer at antibody concentrations of 150 mg/mL and 100 mg/mL. Antibody 5 deviated slightly from Antibodies 1-3 at higher concentrations, though still demonstrated lower % HMW in Arg-Glu at 30° C. but higher levels at 40° C. at 150 mg/mL and 100 mg/mL. The benefits of Arg-Glu in Antibody 4 appear to be protein concentration and temperature dependent, with reversal of % HMW trends observed at 150 mg/mL and 10) mg/mL compared to 200 mg/mL.

As shown in Table 12, the viscosity results suggest that Arg-Glu formulation lowers viscosity at highest protein concentration for most mAbs compared to the Control DF Buffer. Antibody 1 results indicate that viscosity can be maintained or lowered in the Arg-Glu formulation at a slightly higher protein concentration compared to the Control DF buffer formulation.

### Example 3

This example describes formulations comprising a high concentration of Antibody 1.

Additional studies were carried out to develop an isotonic, low viscosity formulation comprising a high concentration of Antibody 1. As in Example 1, the formulations were designed to demonstrate suitable stability, as characterized by low initial high molecular weight (HMW) species formation, as well low HMW species formation upon storage.

Four formulations (A-D) were prepared by diafiltering an initial solution comprising Antibody 1 (100 mg/mL), acetate, and sucrose against a diafiltration (DF) buffer described in Table 13. A total of 10 buffer changes (10 diavolumes) were carried out to achieve a complete buffer exchange. The buffer-exchanged Antibody 1 solution was then concentrated to a concentration >150 mg/mL and was then diluted to achieve the target concentration of 150 mg/mL using the same DF buffer used in the diafiltration. Polysorbate 80 (PS80) was added to a final concentration of 0.01% (w/v). The target pH of each formulation was 5.0.

TABLE 13

| Formulation | | DF Buffer | Target |
|---|---|---|---|
| No. | DF Buffer | pH | pH* |
| A | 20 mM acetate | 4.41 | 5.0 |
| | 8% sucrose | | |
| B | 136 mM L-arginine base | 4.94 | 5.0 |
| | 159 mM glutamic acid | | |
| C | 20 mM acetate | 4.44 | 5.0 |
| | 230 mM proline | | |
| D | 20 mM acetate | 4.41 | 5.0 |
| | 110 mM proline | | |
| | 4% sucrose | | |

*Actual measured pH values ranged from 4.95 to 5.09.

The formulations were evaluated for stability, viscosity, and other physical characteristics as essentially described in Example 1. The osmolality for each formulation was measured and found to be within a range of about 275 mOsm/kg to about 330 mOsm/kg. Viscosity was tested at 5° C. and 25° C. at 150 mg/mL and 165 mg/mL. The viscosity goal of <I0 cP at 25° C. was met by all formulations except for Formulation A at 165 mg/mL. A summary of the viscosity results is provided in Table 14.

TABLE 14

| | Viscosity | Viscosity | Viscosity | Viscosity |
|---|---|---|---|---|
| | at 5° C. | at 5° C. | at 25° C. | at 25° C. |
| Formulation | (150 mg/mL) | (165 mg/mL) | (150 mg/mL) | (165 mg/mL) |
| A | 15.7 cP | 22.3 cP | 8.3 cP | 11.4 cP |
| B | 12.6 cP | 16.6 cP | 6.9 cP | 9.4 cP |
| C | 12.1 cP | 16.2 cP | 6.4 cP | 8.9 cP |
| D | 13.1 cP | 17.8 cP | 7.1 cP | 9.4 cP |

As shown in Table 14, Formulations B-D met the viscosity goal at both concentrations (150 mg/mL and 165 mg/mL).

The stability of the formulations was evaluated by measuring HMWS levels via SE-UHPLC before and after 13 weeks of storage at −30° C. or before and after three freeze-thaw (F/T) cycles (frozen temperature: −30° C.; thawed temperature: room temperature) as essentially described in Example 1. All formulations demonstrated stability as evidenced by <0.5% increase in HMWS after the 13-week storage or after the FIT cycles. Formulations A, B, and D demonstrated the greatest stability as only a 0.2% increase in HMWS was observed after the 13-week storage or after the F/T cycles. As a liquid, Formulations B and C demonstrated the strongest stability profile across many assays, including SEC, CEX, CE, and HIAC. Subvisible particle analyses carried out by HIAC demonstrated that Formulations B and C were essentially particle free. Formulation B had only 416 particles (≥10 μm sized) at t=0 and only 64 particles at t=13 weeks. Formulation C had only 208 particles (≥10 μm sized) at t=0 and only 526 particles at t=13 weeks.

Taken together, while all formulations demonstrated high stability, Formulation B met all design goals for stability and viscosity. Formulation C also demonstrated advantages over other formulations including high stability, low viscosity and low subvisible particle counts.

### Example 4

This example demonstrates the suitability of the presently disclosed formulations for long term storage stability.

Samples of the control and test formulations comprising 100, 150, or 200 mg/mL of one of Antibodies 1-5 described in Example 2 were stored for 59 weeks at −30° C. or 5° C. As described in Example 2, the formulations were prepared as essentially described in Example 1. The components of the test DF buffer and the five control DF buffers are summarized in Table 10. PS80 was added after UF/DF to achieve a final concentration of 0.01% (w/v). The storage stability of each formulation was tested as essentially described in Example 1. A summary of the results for each antibody expressed as % HMW is provided below in Table 15.

TABLE 15

| | Antibody | Test vs | | t = 59 wks | t = 59 wks |
|---|---|---|---|---|---|
| Antibody | Concentration | Control | t = 0 | at −30° C. | at 5° C. |
| 1 | 200 | Control | 2.2 | 2.2 | 3.4 |
| 1 | 200 | Test | 1.6 | 1.7 | 2.7 |
| 1 | 150 | Control | 1.9 | 1.9 | 2.9 |
| 1 | 150 | Test | 1.4 | 1.4 | 2.4 |
| 1 | 100 | Control | 1.4 | 1.5 | 2.1 |
| 1 | 100 | Test | 1.2 | 1.1 | 1.9 |
| 2 | 200 | Control | 0.7 | 0.9 | 1.0 |
| 2 | 200 | Test | 0.4 | 0.5 | 0.7 |
| 2 | 150 | Control | 0.5 | 0.6 | 0.7 |
| 2 | 150 | Test | 0.3 | 0.4 | 0.6 |
| 2 | 100 | Control | 0.4 | 0.4 | 0.5 |
| 2 | 100 | Test | 0.3 | 0.3 | 0.4 |
| 3 | 200 | Control | 1.6 | 1.6 | 2.1 |
| 3 | 200 | Test | 0.8 | 1.0 | 1.1 |
| 3 | 150 | Control | 1.4 | 1.5 | 1.9 |
| 3 | 150 | Test | 0.8 | 0.9 | 1.0 |
| 3 | 100 | Control | 1.0 | 1.1 | 1.2 |
| 3 | 100 | Test | 0.6 | 0.7 | 0.7 |
| 4 | 200 | Control | 1.9 | 2.0 | 2.1 |
| 4 | 200 | Test | 1.8 | 1.9 | 1.9 |
| 4 | 150 | Control | 1.8 | 1.9 | 1.9 |
| 4 | 150 | Test | 1.7 | 1.7 | 2.0 |
| 4 | 100 | Control | 1.7 | 1.7 | 1.6 |
| 4 | 100 | Test | 1.7 | 1.7 | 1.9 |
| 5 | 150 | Control | 1.3 | 1.3 | 1.6 |
| 5 | 150 | Test | 1.0 | 1.0 | 1.3 |
| 5 | 100 | Control | 1.1 | 1.1 | 1.3 |
| 5 | 100 | Test | 0.9 | 0.9 | 1.2 |

These results show that the test formulations comprising L-arginine base and L-glutamic acid have lower starting HMW by SEC at t=0 and maintains a lower amount of HMW even after 59 weeks at both storage temperatures (−0° C. an 5° C.) relative to the control formulations lacking L-arginine base and L-glutamic acid. These data support the feasibility of a long shelf-life of the antibody formulations, e.g., 2+ years at −30° C. or 2+ years at 2° C. to 8° C.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms including the indicated component(s) but not excluding other elements (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

1. A liquid composition comprising (a) an anti-IL-15 antibody at a concentration greater than about 100 mg/mL, (b) about 70 mM to about 210 mM arginine base, (c) about 80 mM to about 240 mM glutamate, and (d) a surfactant, wherein the pH of the liquid composition is about 4.5 to about 5.5.

2. The liquid composition of claim 1, comprising:
about 100 mM to about 170 mM arginine base; and/or
about 120 mM to about 200 mM glutamate.

3. The liquid composition of claim 2, comprising:
about 120 mM to about 150 mM arginine base; and/or
about 140 mM to about 175 mM glutamate.

4.-7. (canceled)

8. The liquid composition of claim 1, wherein arginine and glutamate are the only amino acids present in the liquid composition.

9. (canceled)

10. (canceled)

11. A liquid composition comprising (a) an anti-IL-15 antibody at a concentration greater than about 100 mg/mL, (b) about 115 mM to about 345 mM proline, (c) a buffer, and (d) a surfactant, wherein the pH of the liquid composition is about 4.5 to about 5.5.

12. The liquid composition of claim 11, comprising about 170 mM to about 290 mM proline.

13. The liquid composition of claim 12, comprising about 200 mM to about 255 mM proline.

14. (canceled)

15. The liquid composition of claim 11, wherein proline is the only amino acid present in the composition.

16. The liquid composition of claim 1, comprising about 1 mM to about 100 mM buffer, wherein the buffer is selected from the group consisting of succinate, glutamate, histidine, and acetate.

17.-19. (canceled)

20. The liquid composition of claim 1, comprising not more than 0.001% (w/v) of a sugar, sugar alcohol, or citrate and/or not more than 0.001% (w/v) disaccharide.

21.-24. (canceled)

25. The liquid composition of claim 1, comprising about 110 mg/mL to about 200 mg/mL anti-IL-15 antibody.

26.-31. (canceled)

32. The liquid composition of claim 1, wherein the anti-IL-15 antibody is an IgG₁ antibody.

33. The liquid composition of claim 32, comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6.

34. The liquid composition of claim 32, comprising a HC variable region of SEQ ID NO: 7 and a LC variable region of SEQ ID NO: 8.

35. The liquid composition of claim 32, comprising a HC of SEQ ID NO: 9 and a LC of SEQ ID NO: 10.

36. (canceled)

37. (canceled)

38. The liquid composition of claim 1, wherein;
the surfactant is polysorbate 20 (PS20) or polysorbate 80 (PS80) or a mixture thereof; and
the surfactant is present at a concentration of about 0.005% (w/v) to about 0.025% (w/v).

39.-48. (canceled)

49. The liquid composition of claim 1, wherein:
the viscosity of the liquid composition is less than 50 cP at 25° C., 1000 s⁻¹; and/or
the composition has an osmolality in a range of about 200 mOsm/kg to about 500 mOsm/kg; and/or
less than 5% of the antibody is degraded after storage at 2° C. to 8° C. for at least or about 12 months or about 24 months to about 36 months, as determined by Size Exclusion Chromatography (SEC).

50. The liquid composition of claim 49, wherein;
the viscosity of the liquid composition is less than 30 cP at 25° C., 1000 s⁻¹; and/or
the composition has an osmolality in a range of about 250 mOsm/kg to about 350 mOsm/kg; and/or
less than 5% of the antibody is degraded after storage at 2° C. to 8° C. for about 30 to about 40 months, as determined by SEC.

51. The liquid composition of claim 50, wherein:
the viscosity of the liquid composition is less than 20 cP at 25° C., 1000 s⁻¹; and/or
the composition is isotonic; and/or
less than 5% of the antibody is degraded after storage at 2° C. to 8° C. for about 36 to about 40 months, as determined by SEC.

52.-58. (canceled)

59. A liquid composition comprising, per mL of the liquid composition,
(i) about 135 mg to about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6,
(ii) about 21 mg to about 26 mg arginine base,
(iii) about 21 mg to about 26 mg glutamate, and
(iv) about 0.01% (w/v) polysorbate 80 (PS80), wherein the liquid composition has a pH of about 4.5 to about 5.5.

60. A liquid composition comprising, per mL of the liquid composition,
(i) about 135 mg to about 165 mg of an anti-IL-15 antibody comprising a Heavy Chain (HC) Complementarity-Determining Region (CDR) 1 of SEQ ID NO: 1, a HC CDR2 of SEQ ID NO: 2, a HC CDR3 of SEQ ID NO: 3, a Light Chain (LC) Complementarity-Determining Region (CDR) 1 of SEQ ID NO: 4, a LC CDR2 of SEQ ID NO: 5, and a LC CDR3 of SEQ ID NO: 6,
(ii) about 23 mg to about 30 mg proline,
(iii) about 0.5 mg to about 2 mg acetate, and
(iv) about 0.01% (w/v) polysorbate 80 (PS80), wherein the liquid composition has a pH of about 4.5 to about 5.5.

61. A method of preparing a liquid composition comprising greater than about 100 mg/mL of an anti-IL-15 antibody, said method comprising (a) combining the antibody with a diafiltration (DF) buffer comprising about 70 mM to about 210 mM arginine base and about 80 mM to about 240 mM glutamic acid, and (b) adding a surfactant.

62.-73. (canceled)

74. An article of manufacture comprising the liquid composition of claim 1, optionally, comprising about 1 mL to about 5 mL of the liquid composition.

75. A pre-filled syringe comprising the liquid composition of claim 1, optionally, comprising about 1 mL to about 5 mL of the liquid composition.

76. A vial comprising the liquid composition of claim 1, optionally, comprising about 1 mL to about 5 mL of the liquid composition.

77. An autoinjector comprising the liquid composition of claim 1.

78. A method of treating a disease in a subject, comprising administering to the subject a liquid composition of claim 1 in an amount effective to treat the disease.

79. (canceled)

80. The method of claim 78, wherein the disease is a celiac disease.
